(19) United States
(12) Patent Application Publication
(10) Pub. No.: US 2024/0026334 A1
(43) Pub. Date: Jan. 25, 2024

(54) DNA-COLLAGEN COMPLEXES AND MAGNETOELECTRIC JANUS MATERIALS FOR BIOMEDICAL APPLICATIONS

(71) Applicant: UNIVERSITY OF FLORIDA RESEARCH FOUNDATION, Gainesville, FL (US)

(72) Inventors: Josephine ALLEN, Gainesville, FL (US); Jennifer S. ANDREW, Gainesville, FL (US); Noah D. FERSON, Gainesville, FL (US); Bryan D. JAMES, Gainesville, FL (US)

(21) Appl. No.: 18/044,094

(22) PCT Filed: Sep. 10, 2021
(86) PCT No.: PCT/US2021/049833

§ 371 Date: Mar. 6, 2023

Related U.S. Application Data
(60) Provisional application No. 63/077,229, filed on Sep. 11, 2020.

(51) Int. Cl. C12N 13/00; C12N 5/00; C12N 15/115

(52) U.S. Cl. CPC C12N 13/00; C12N 5/0068; C12N 15/115; C12N 2529/00; C12N 2533/54; C12N 2310/16; C12N 2310/3513

(57) ABSTRACT

Disclosed herein are compositions comprising magnetoelectric composite materials and collagen, along with uses and kits thereof. Described herein is the use of DNA aptamer assemblies of varying DNA length, structure, and sequence to both bind to collagen and other proteins, to then act as a biocompatible, degradable, reversible, or permanent 3D crosslinkers between proteins, and to service as a biologically functional material when using the appropriate aptamer sequence. Therefore, disclosed herein are compositions comprising collagen fibers crosslinked with DNA aptamers. Also disclosed are devices and implants made from or coated with collagen fibers crosslinked with DNA aptamers. Also disclosed are methods of making collagen fibers. Also disclosed are kits for producing collagen fibers. Also disclosed herein are compositions DNA aptamers in a collagen fiber matrix that stabilizes the DNA aptamer.

### CROSS-REFERENCE TO RELATED APPLICATIONS

This application claims the benefit of and priority to U.S. Provisional Patent Application No. 63/077,229 entitled “DNA-COLLAGEN COMPLEXES AND MAGNETOELECTRIC JANUS MATERIALS FOR BIOMEDICAL APPLICATIONS” filed on Sep. 11, 2020, which is expressly incorporated by reference as if fully set forth herein in its entirety.

### STATEMENT REGARDING FEDERALLY SPONSORED RESEARCH OR DEVELOPMENT

This invention was made with government support under Grant Numbers 1453098 and 1410564, awarded by the National Science Foundation. The government has certain rights in the invention.

### SEQUENCE LISTING

This application contains a sequence listing filed in electronic form as an ASCII.txt file entitled “222111_2020 Sequence Listing_ST25.txt”, created on Sep. 10, 2021 and having a size of 3 kb. The content of the sequence listing is incorporated herein in its entirety.

### BACKGROUND

Crosslinking of collagen is an effective method to modify the stability of collagen compositions and materials and to optimize their mechanical and structural properties. Crosslinked collagen materials are used extensively in various medical and industrial applications. For example, crosslinked collagen materials are used to replace or augment hard or soft connective tissue, such as skin, tendons, cartilage, bone, and interstitium. Crosslinked collagen materials have been implanted surgically, and numerous injectable crosslinked collagen formulations are currently available for various cosmetic applications. Toxic chemicals typically are used for crosslinking collagen and additives (growth factors, small molecules, drugs) are incorporated to achieve biological responses. However, these can lead to detrimental off-target effects. However, there remains a need in the art for improved methods of producing crosslinked collagen materials and endowing them with greater functionality at the same time.

### SUMMARY

Oligonucleotides are capable of binding protein. A specific type of oligonucleotide structure is DNA aptamer. These are single stranded oligonucleotide sequences, which are capable of forming tertiary structures. This enables them to upon targeting and purification have extremely high specificity for a specific biological structure. Moreover, our group has shown that DNA aptamers can activate cell signaling receptors, rather than simply passively attaching to the receptor. Specifically, relevant are extracellular matrix proteins, notably collagen. This protein forms fibers in solution, which are typically crosslinked to form a 3D network by chemical means such as with cytotoxic glutaraldehyde. DNA is capable of forming a complex with collagen, which initiates and promotes collagen fiber formation. These fibers are then DNA-collagen complexes. We have found that DNA aptamers are capable of forming these complexes and the fiber properties are dependent on both the aptamer sequence, geometry, and relative concentration. These fibers begin to form spontaneously upon combining DNA and collagen solutions. DNA aptamers are also capable of being conjugated together to form 3D assemblies.

Disclosed herein is the use of DNA aptamer assemblies of varying DNA length, structure, and sequence to both bind to collagen and other proteins, to then act as a biocompatible, degradable, reversible, or permanent 3D crosslinkers between proteins, and to service as a biologically functional material when using the appropriate aptamer sequence.

Therefore, disclosed herein are compositions comprising collagen fibers crosslinked with a plurality of one or more DNA aptamers. Also disclosed are devices and implants made from or coated with collagen fibers crosslinked with a plurality of one or more DNA aptamers. Also disclosed are methods of making collagen fibers. Also disclosed are kits for producing collagen fibers. Also disclosed herein are compositions comprising a plurality of one or more DNA aptamers in a collagen fiber matrix that stabilizes the DNA aptamers.

In some embodiments, at least one of the one or more DNA aptamers selectively binds a growth factor or cytokine. In some embodiments, at least one of the one or more DNA aptamers selectively binds a cell receptor, such as a stem cell receptor. In some embodiments, at least one of the one or more DNA aptamers selectively binds an extracellular matrix protein.

In some embodiments, the DNA aptamers comprise from 15 to 100 nucleotides, including 15, 16, 17, 18, 19, 20, 25, 30, 35, 40, 45, 50, 60, 70, 80, 90, 100 nucleotides.

In some embodiments, the DNA aptamers comprise from 1 to 20 stem loops, including 1, 2, 3, 4, 5, 6, 7, 8, 9, 10, 11, 12, 13, 14, 15, 16, 17, 18, 19, or 20 stem loops, such as 1 to 10 stem loops, or 1 to 5 stem loops.

In some embodiments, the plurality of one or more DNA aptamers comprises 2 or more DNA aptamer sequences, including 2 to 4 DNA aptamer sequences, connected by a linker molecule to form an aptamer assembly.

In some embodiments, the collagen comprises type I collagen, type II collagen, type III collagen, type V collagen, type XI collagen, or any combination thereof.

The details of one or more embodiments of the invention are set forth in the accompanying drawings and the description below. Other features, objects, and advantages of the invention will be apparent from the description and drawings, and from the claims.

Described herein are compositions. In embodiments, compositions described herein comprise a collagen material and a magnetoelectric composite material. In embodiments, the collagen material can comprise collagen fibers crosslinked with a plurality of one or more DNA aptamers. In embodiments, at least one of the one or more DNA aptamers selectively binds a growth factor or cytokine. In embodiments, at least one of the one or more DNA aptamers selectively binds a cell receptor. In embodiments, the cell receptor is a cell surface receptor and is a signaling receptor, a stem cell receptor or growth factor receptor. Such receptors can be receptors of neurons, glia, vasculature, or endothelial cells. In embodiments, at least one of the one or more DNA aptamer selectively binds an extracellular matrix protein. In embodiments, DNA aptamers can comprise from 15 to 100 nucleotides. In embodiments, DNA aptamers comprise from 1 to 5 stem loops. In embodiments, a plurality of one or more DNA aptamers comprises 2 to 4 DNA aptamer sequences connected by a linker molecule to form an aptamer assembly. In embodiments, the collagen comprises type I collagen, type II collagen, type III collagen, type V collagen, type XI collagen, or any combination thereof. In embodiments, the magnetoelectric composite material comprises a piezoelectric composite material or a magnetorestrictive compositive material, individually or in combination. In embodiments, the magnetoelectric composite material comprises BaTiO₃ and CoFe2O₄. In embodiments, the magnetoelectric composite material comprises electrospun fibers. In embodiments, the electrospun fibers have a length of about 1 μm to about 100 μm. In embodiments, the electrospun fibers have a diameter of about 100 nm to about 1000 nm.

Described herein are methods of tissue culture. In embodiments, a method of tissue culture comprises providing a composition as described herein; and culturing one or more cells in the presence of the composition, a magnetic field, and an electric field. In embodiments, the magnetic field can have a field strength of about 0 mT to about 5 mT. In embodiments, the electric field can have a field strength of about 0 to about 500 mV/mm. In embodiments, the one or more cells comprise neuronal cells, neuronal progenitor cells, epithelial cells, fibroblasts, osteoclasts, osteoblasts, or muscle cells.

Described herein are kits for tissue culture. In an embodiment, a kit for tissue culture can comprise collagen monomers and an magnetoelectric composite material. In embodiments, kits as described herein further comprise one or more DNA aptamers. In embodiments, at least one of the DNA aptamers of kits as described herein selectively binds a growth factor or cytokine. In embodiments, at least one of the DNA aptamers of kits as described herein selectively binds a cell receptor. In embodiments of kits as described herein, the cell receptor is a stem cell receptor or nerve growth factor receptor. In embodiments, at least one of the DNA aptamers of kits as described herein selectively binds an extracellular matrix protein. In embodiments, the DNA aptamers of kits as described herein comprise from 15 to 100 nucleotides. In embodiments, DNA aptamers of kits as described herein comprise from 1 to 5 stem loops. In embodiments of kits as described herein, the plurality of one or more DNA aptamers comprises 2 to 4 DNA aptamer sequences connected by a linker molecule to form an aptamer assembly. In embodiments of kits as described herein, the collagen can comprise type I collagen, type II collagen, type III collagen, type V collagen, type XI collagen, or any combination thereof. In embodiments of kits as described herein, the magnetoelectric composite material can comprise a piezoelectric composite material or a magnetorestrictive compositive material, individually or in combination. In embodiments of kits as described herein, the magnetoelectric composite material can comprise BaTiO₃ and CoFe2O₄. In embodiments of kits as described herein, the magnetoelectric composite material can comprise electrospun fibers. In embodiments of kits as described herein, the electrospun fibers can have a length of about 1 μm to about 100 μm. In embodiments of kits as described herein, the electrospun fibers can have a diameter of about 100 nm to about 1000 nm.

### DESCRIPTION OF DRAWINGS

FIG. 1 shows a panel of images comparing ssDNA-collagen fibers formed using various relative amounts of ssDNA and collagen. Fibers formed for solutions of 57 and 82% mass fraction collagen but not for the 92% mass fraction collagen solution.

FIGS. 2A to 2D show a normality plot (FIG. 2A), a residual versus fitted value plot (FIG. 2B), a histogram of fit residuals (FIG. 2C), and a residual order plot (FIG. 2D) all of which indicate that the 3^{rd} order polynomial regression was an appropriate fit.

FIGS. 3A and 3B show regression model effects. The main effects plot shows that fiber formation is dependent on the volume fraction of collagen in solution with a maximum around 0.2-0.4. In addition, there is little change in turbidity over time indicating that fibers formed very rapidly upon ssDNA and collagen mixing (FIG. 3A). These trends are independent as shown by the interaction plot (FIG. 3B).

FIGS. 4A and 4B show ssDNA localizes and is present in the fibers as indicated by red fluorescence from ethidium bromide homodimer staining.

FIGS. 5A and 5B show ssDNA binding to collagen increases with decreasing amount of collagen in solution relative to the amount of ssDNA in solution (FIG. 5A). In addition, the amount of ssDNA binding increases as more collagen is available in solution (FIG. 5B).

FIG. 6 is an illustration of example aptamers disclosed herein showing SEQ ID NOs:6-8.

FIG. 7 shows sequences and predicted structures of random 15, 33, 45, and 90 nucleotide (nt) ssDNA oligomers (SEQ ID NO:2,3,4, and 5, respectively). Predicted structures were calculated using the mFold web server.

FIGS. 7 and 8A to 8C show ssDNA oligomers with 15 (SEQ ID NO:2), 33 (SEQ ID NO:3), 45 (SEQ ID NO:4), and 90 (SEQ ID NO:5) nucleotides (nt) and their binding to type I collagen. ssDNA binding to collagen measured as the mass of bound DNA per mass of collagen as a function of mass fraction of DNA in solution (FIG. 8A). ssDNA binding to collagen measured as the moles of bound DNA per mass of collagen as a function of mass fraction of DNA in solution (FIG. 8B). The horizontal bars in (FIG. 8B) represent the range of DNA mass fraction where fiber formation was observed, from the top oligomers were 15, 33, 45 and 90 nt, respectively. When value for maximum binding from (FIG. 8B) of each oligomer was plotted against the inverse of the oligomer molecular weight, the data followed a linear relationship with R²>0.95 (FIG. 8C). ssDNA binding was measured in triplicate. Data is presented as mean±standard deviation.

FIG. 9 shows representative fluorescence microscopy images of immobilized ssDNA-collagen fibers formed ssDNA with lengths of 15, 33, 45, and 90 nucleotides (nt) and different volume fractions of collagen. ssDNA in the fibers was fluorescently labeled using SYBR Safe DNA stain.

FIG. 10 shows properties of BaTiO₃—CoFe₂O₄ Magnetoelectric Janus Fibers.

FIGS. 11A-11B are plots showing length control of magnetoelectric nanowires.

FIG. 12 is a plot of ferrimagnetic properties of BaTiO₃—CoFe₂O₄ composites according to the present disclosure.

FIG. 13 is a plot showing magnetoelectric coupling of BTO-CFO janus composites.

FIGS. 14A and 14B represent magnetoelectric coefficient of arrays of BaTiO₃/CoFe₂O₄Nanowires.

FIG. 15 shows plots of a lock in magnetoelectric measurements.

FIG. 16 illustrates aspects of tissue culture according to the present disclosure.

FIG. 17 is a cartoon of magnetoelectric stimulation according to the present disclosure.

FIGS. 18A-18C are photographs of a reduced-to-practice embodiment of a tissue culture setup according to the present disclosure.

FIGS. 19A-19C are plots showing the effect of magnetoelectric stimulation on toxicity.

FIGS. 20A-20B are plots showing the effect of magnetoelectric stimulation on toxicity.

FIG. 21 shows fluorescent micrographs of culture of cells with collagen, collagen+CFO alone, and collagen plus janus materials with growth media (unstimulated and stimulated) at days 0, 1, 3, and 5.

FIG. 22 shows fluorescent micrographs of culture of cells with collagen, collagen+CFO alone, and collagen plus janus materials with differentiation media (unstimulated and stimulated) at days 0, 1, 3, and 5.

FIGS. 23A-23B are fluorescent micrographs of culture of cells with collagen plus janus materials with differentiation media (unstimulated and stimulated) at day 5.

FIGS. 24A-24B illustrates co-electrospin biphasic Janus type magnetoelectric materials and properties thereof according to the present disclosure.

FIG. 25 is a fluorescent micrograph of culture of cells with collagen plus janus materials with differentiation media (stimulated) at day 5.

FIGS. 26A-26E show phase contrast images of nucleic acid-collagen complex (NACC) fibers in aqueous solution (A), BTO-CFO janus (eJPF) particulate in aqueous solution (B), the association of collagen (COL) and BTO-CFO janus particulate in aqueous solution (C), the association of single-stranded DNA (ssDNA) and BTO-CFO janus particulate in aqueous solution, and the association of nucleic acid-collagen complex fibers (NACCs) and BTO-CFO janus particulate in aqueous solution.

FIGS. 27A-27B are two phase contrast images showing the association of nucleic acid-collagen complex fibers (NACCs) and BTO-CFO janus particulate in aqueous solution.

FIGS. 28A-28D are phase contrast images showing surfaces functionalized with sulfo-SANPAH, the functionalizing species (A), single-stranded DNA (ssDNA) (B), collagen (COL) (C), and nucleic acid-collagen complex fibers (NACCs) (D).

FIGS. 29A-29D are phase contrast images showing surfaces functionalized with sulfo-SANPAH, the functionalizing species (A), single-stranded DNA (ssDNA) (B), collagen (COL) (C), and nucleic acid-collagen complex fibers (NACCs) (D) after exposure to BTO-CFO janus particulate. A-C show random organization. D shows non-random organization and association of the BTO-CFO janus particulate with the NACC fibers (magNACC).

FIGS. 30A-30B are micrographs showing a fluorescence image of the single-stranded DNA (ssDNA) contained in the nucleic acid-collagen complex fibers stained with a fluorescent intercalating DNA dye (A), and showing a phase contrast image of the same location highlighting the non-random organization and association of the BTO-CFO janus particulate with the NACC fibers (magNACC).

FIGS. 31A-31C are micrographs showing a phase contrast image of the of the non-random organization and association of the BTO-CFO janus particulate with the NACC fibers (magNACC) (A), a fluorescence image of the same location of the single-stranded DNA (ssDNA) contained in the nucleic acid-collagen complex fibers stained with a fluorescent intercalating DNA dye (B), and the overlap of image A and B (C).

FIGS. 32A-32C are micrographs and a plot showing that BTO-CFO janus particulate can be aligned using magnetic fields.

FIGS. 33A-33D: FIG. 33A shows a fluorescence image of NACC fibers and FIG. 33B displays a corresponding measurement of Young's modulus. FIG. 33C is a fluorescence image of NAECC fibers and FIG. 33D a corresponding Young's modulus.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “DNA aptamer” refers to a single stranded deoxyribonucleic acid (DNA) whose distinct nucleotide sequence determines the folding of the molecule into a unique three dimensional structure. Aptamers comprising 15 to 120 nucleotides can be selected in vitro from a randomized pool of oligonucleotides (10¹⁴-10¹⁵ molecules). The “DNA aptamer” comprises a degenerate sequence, and can further comprise fixed sequences flanking the degenerate sequence. The term “DNA aptamer” as used herein further contemplates the use of both native and modified DNA bases, e.g. beta-D-Glucosyl-Hydroxymethyluracil.

As used herein, the term “DNA aptamer” refers to an oligonucleotide molecule that binds to a target protein. In some embodiment, the DNA aptamer binds to a specific region or amino acid sequence of the target protein.

As used herein, the term “bind,” the term “binding” or the term “bound” refers to any type of chemical or physical binding, which includes but is not limited to covalent binding, hydrogen binding, electrostatic binding, biological tethers, transmembrane attachment, cell surface attachment and expression.

For purposes of the present invention, the term “oligonucleotide,” the term “polynucleotide,” the term “nucleotide,” and the term “nucleic acid” refer to a molecule comprised of two or more deoxyribonucleotides or ribonucleotides, and usually more than ten. The exact size of an oligonucleotide will depend on many factors, which in turn depends on the ultimate function or use of the oligonucleotide. The oligonucleotide may be generated in any manner, including chemical synthesis, DNA replication, reverse transcription, or a combination thereof. When present in a DNA form, the oligonucleotide may be single-stranded (i.e., the sense strand) or double-stranded.

For purposes of the disclosed invention, the term “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

For purposes of the disclosed invention, the term “residue,” the term “amino acid residue,” or the term “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

Discussion

Described herein are compositions, methods, and kits related to compositions comprising magnetoelectric composite materials and collagens. In embodiments, described herein are compositions, methods, and kits related to compositions comprising magnetoelectric composite materials, collagens, and nucleic acids.

In certain aspects of the present disclosure, DNA-collagen complex self-assemblies have been combined with ceramic Janus materials to form a composite material that has potential biomedical applications. Short, single-stranded DNA with collagen was first combined at the appropriate ratio and concentration to form DNA-collagen complex fibers. In an embodiment, subsequently, the fibers can be exposed to an aqueous dispersion of powdered cobalt ferrite/barium titanate Janus fibers (also referred to herein as magnetoelectric composite materials). These fibers are electroactive and can be used to locally generate electric fields. The Janus fibers incorporated into the embodiment of DNA-collagen fibers can form a three species composite material of DNA, collagen, and Janus fiber. These composite materials have tremendous room for customizability by varying the DNA species, the collagen species, and the Janus fiber species. DNA-collagen complex materials can also be formed into not only fibers but also nanoparticles and 3D hydrogels.

Nucleic acids as described herein can comprise single or double stranded DNA. Nucleic acids as described herein can comprise DNA aptamers.

Additional aspects of collagens and magnetoelectric composite materials are described below.

DNA Aptamers

Disclosed herein are DNA aptamers that can be used to crosslink collagen into fibers. The DNA sequence used to produce the aptamer can be selected using routine methods based on desired characteristics, such as protein binding.

DNA aptamers are short, single-stranded DNA oligonucleotides capable of specific binding to defined targets. The advent and success of SELEX technology in 1990s may be attributed to the feasibility to chemically synthesize pools of random oligonucleotides, the availability of the polymerases for nucleic acid amplification, as well as the improvement in sequencing techniques. The molecular recognition between aptamers and their corresponding targets relies on the three-dimensional conformations of the aptamers, hence the specific nucleic acid sequences. By substituting just a few nucleotides, the conformation of an oligonucleotide may change. Consequently, the structural diversity of a DNA or RNA pool containing combinatorial sequences may be infinitely expanded, thereby creating panels of aptamers for a wide variety of binding targets. The evolution process for selecting DNA aptamers typically covers the following steps: 1) chemical synthesis of a combinatorial oligonucleotide library having 10¹³-10¹⁶ single stranded nucleic acid molecules, 2) exposure of the library to the targets to differentiate binding strands from spectators, 3) extraction and amplification of eluted survivors, 4) enrichment of the stronger survivors by iterative binding to targets and by involving counter selection if necessary, and, finally, 5) sequencing to identify individual candidates.

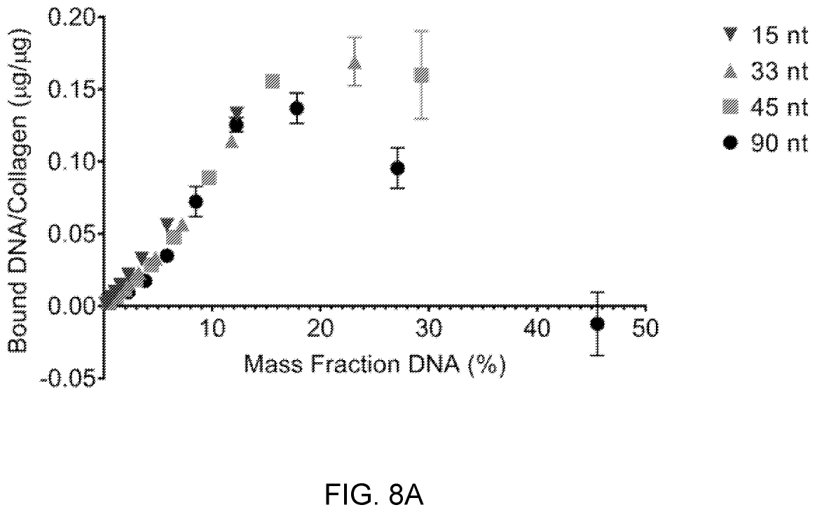

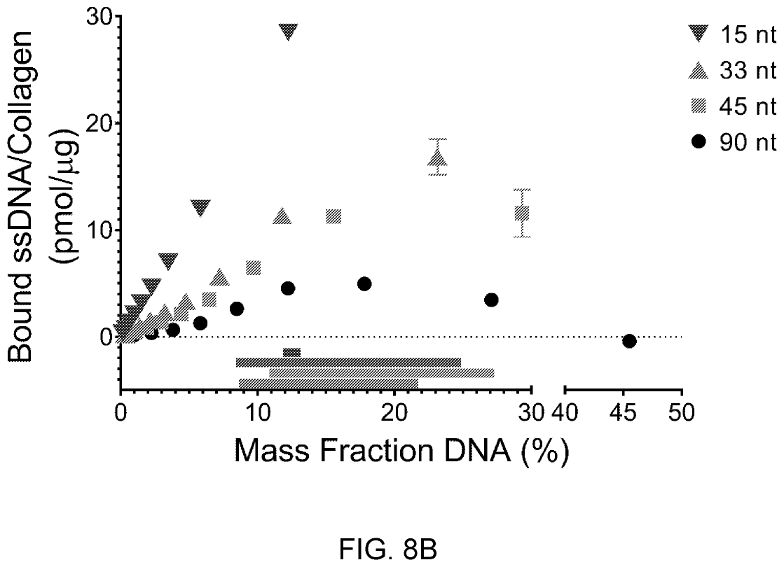

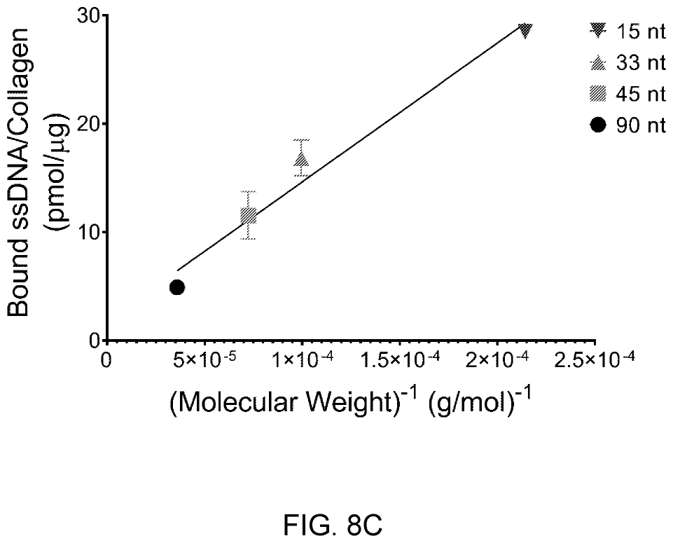

The SELEX process (systematic evolution of ligands by exponential enrichment) for engineering DNA aptamer sequences generates several potential candidates of varying length. As the disclosed data shows, fiber formation is dependent on both ssDNA length and the relative amounts of ssDNA and collagen in solution. Thus, the choice of sequence from the SELEX process is important as the ideal recipe for fiber formation will be different for each candidate sequence. Fibers form above a threshold binding value of 0.05 μg ssDNA/μg collagen, but also required the appropriate amount of ssDNA and collagen in solution (8-30% mass fraction DNA in solution) (FIG. 8B). Too much of either ssDNA or collagen in solution compared to the other inhibits fiber formation due to self-aggregation. More molecules of ssDNA bind with collagen as the sequence length decreases (FIG. 8C). This implies that more individual oligomers of ssDNA are present in the fibers for shorter sequences. Therefore, when fibers are formed using a DNA aptamer, the number of moles of aptamer oligomer per mass of collagen is greater for shorter sequences. Thus, fibers formed using a shorter DNA aptamer have a greater capacity for binding to the DNA aptamer target. This enables DNA aptamer targeting by the fibers to be tuned by varying the DNA aptamer sequence length. In addition, fiber formation requires the ssDNA and collagen to be mobile i.e. in solution. Fibers do not form when either component is immobilized to a surface and exposed to the other in solution. Thus, the fibers must first be synthesized and then immobilized for surface modification applications.

The length of the DNA aptamer comprising the sequence (i) or (ii) or the sequence (I) or (II) (hereafter, simply referred to as the “DNA aptamer according to the present invention”) is, for example, 150 mer or shorter, 140 mer or shorter, 130 mer or shorter, 120 mer or shorter, or 110 mer or shorter, and preferably 100 mer or shorter, 90 mer or shorter, 80 mer or shorter, 70 mer or shorter, 60 mer or shorter, or 50 mer or shorter.

The DNA aptamer according to the present invention may arbitrarily comprise a base analog, another artificial base, another modified base, or the like, in addition to Ds.

The DNA aptamer according to the present invention may be modified with the addition of other substances, such as polyethylene glycol (PEG) (e.g., a PEG polymer of about 20 to 60 kDa), an amino acid, a peptide, inverted dT, a lipid, a dye, a fluorescent substance, an enzyme, a radioactive substance, and biotin. Such substance may be linked via a known linker, if needed. Examples of linkers that can be used herein include a nucleotide linker, a peptide linker, and a linker containing a disulfide bond. It is generally known that a half-life of the DNA aptamer is extended by conjugating PEG to the DNA aptamer.

A method for producing the DNA aptamer according to the present invention is not particularly limited. A method known in the art may be employed. For example, the DNA aptamer according to the present invention can be chemically synthesized based on the sequences indicated above in accordance with a known solid-phase synthesis method. Regarding a method of chemical synthesis of nucleic acids, see, for example, Current Protocols in NucleicAcid Chemistry, Volume 1, Section 3. Many life science manufacturers (e.g., Takara Bio Inc. and Sigma-Aldrich Corporation) provide contract manufacturing services concerning such chemical synthesis, and such services may be used. A DNA aptamer may be prepared by synthesizing several fragments based on the DNA aptamer sequence and then ligating the fragments via, for example, intramolecular annealing or ligation by a ligase.

The DNA aptamer according to the present invention prepared via chemical synthesis is preferably purified by a method known in the art before use. Examples of methods of purification include gel purification, affinity column purification, and HPLC.

Protein Targets

The disclosed aptamers are in some embodiments able to bind a protein of interest. Examples protein targets include growth factors, cytokines, cell receptors, and extracellular matrix proteins.

Examples of pathogen proteins for which DNA aptamers have been developed include Anthrax Protective Antigen, bipd (type iii secretion protein), bope (type iii secretion protein), Botulinum neurotoxin type A, bpsI2748 (putative oxidoreductase), clostridium difficil toxin a, clostridium difficil toxin b, ETEC K88 fimbriae protein, Francisella tularensis subspecies (subsp) japonica bacterial antigen, Iron-regulated surface determinant a, Iron-regulated surface determinant b, Iron-regulated surface determinant c, Iron-regulated surface determinant h, Leishmania infantum H2A antigen, Leishmania infantum KMP-11, mannose-capped lipoarabinomannan, microcystin-LA, microcystin-LR, microcystin-YR, and -LA, Mycobacterium avium sp. Paratuberculosis Major Antigens, Mycobacterium tuberculosis cfp10, Mycobacterium tuberculosis esat6, Mycobacterium tuberculosis esxg, Mycobacterium tuberculosis methionyl-tRNA synthetase (MRS), Mycobacterium tuberculosis mpt64 protein, Mycobacterium tuberculosis polyphosphate kinase, Plasmodium falciparum erythrocyte membrane protein 1, Plasmodium lactate dehydrogenase, Protein A, Salmonella typhimurium ompc, Staphylococcus aureus clumping factor a, Staphylococcus aureus clumping factor b, Staphylococcus aureus Enterotoxin B, Staphylococcus aureus enterotoxin c1, Staphylococcus aureus Protein A (SpA), Staphylococcus aureus α Toxin, T. cruzi excreted secreted antigens, Type IVB Pili, and Ustilago maydis RNA-binding protein Rrm4.

Examples of viral proteins for which DNA aptamers have been developed include Alfalfa Mosaic virus RNA-coat protein complex, bacteriophage ff gene 5, dengue-2 virus envelope protein domain iii, Ebola Virus VP35 interferon inhibitory domain, Foot-and-mouth disease virus RNA-dependent RNA polymerase, gp130, HBV capsid, HBV core protein, HBV recombinant truncated P protein, HBV surface antigen, HCV core antigen, HCV Envelope Glycoprotein E2, HCV nonstructural protein 3 protease, HCV NS2 protein, HCV NS5A, HCV ns5b replicase, HCV RNA-Dependent RNA Polymerase, HES 1 protein icp27, HIV gp120, HIV integrase, HIV LTR, HIV Nucleocapsid Protein, HIV Tat, HIV-1 gag, HIV-1 Reverse transcriptase, HIV-1 Tar RNA, HPV-16 E7 Oncoprotein, HSV gd protein, HSV US11, HTLV-I tax, Human norovirus (Gii.4) capsid P domain, Human norovirus capsid protein vp1, Influenza A Hemagglutinin, Influenza A NS1 protein, influenza A polymerase PA subunit, influenza protein e, MS2 coat protein, papillomavirus 16 e7 oncoprotein, RABV glycoprotein, rex, Rift Valley fever virus N protein Pool, sars-cov, sars-cov nucleocapsid protein, severe acute respiratory syndrome (sars) coronavirus ntpase/helicase, SP6 RNA polymerase, and Venezuelan equine encephalitis virus capsid protein.

Examples of toxins for which DNA aptamers have been developed include Colicin E3, gliadin peptide, Ricin A-chain, shiga toxin, t-2 toxin, tetanus toxoid, type a botulinum neurotoxin, α-bungarotoxin snake venom, and β-bungarotoxin.

Examples of prions for which DNA aptamers have been developed include bovine prion protein, cellular prion protein, mouse prion, recombinant human (rhu) cellular prion protein (PrPC) 23-231, and Syrian golden hamster prion protein rPrP23-231.

Examples of mammalian proteins for which DNA aptamers have been developed include 4-1BB, Acetohydroxyacid synthase, activated protein c, AGE-human serum albumin, AlkB, Alzheimer's Disease Amyloid Peptide, AML1 Runt domain, AMPA receptor, amyloid-like fibrils, angiogenin, angiopoietin 1, angiopoietin 2, anti-MPT64 antibodies, anti-NF-kB p65, antivesicular stomatitis virus polyclonal antibodies, ApoE, arginine-rich motif (ARM) model peptide, ATR/TEM8 Von Willebrand factor type A (VWA), B-cell-activating factor-receptor, B52 (SR protein RNA splicing), b7, basic fibroblast growth factor, Bcs1, bovine catalase, bovine factor ix, bovine follicle-stimulating hormone α subunit, bovine serum albumin, bovine thrombin, brain natriuretic peptide (bnp), C-C chemokine receptor type 5, c-Met, C-reactive protein, c-telopeptide (ctx) of human type i bone collagen, c4, c7, calsenilin, cardiac troponin i (ctni), cathepsin E, cd133, CD16, cd18, CD28, CD30, cd31, CD40, cd44, Cdc42, CGRP peptide, CHK2, cholesterol esterase, cJun/cJun, complement factor c5a, Connective tissue growth factor, crd11, CTAP III/NAP2, ctla-4, CYT-18, cytochrome c, cytochrome P450 51A1, cytoplasmic tail of BACE, DC-SIGN protein, DNA binding domain of TCF-1, DNA polymerase b, E-Selectin, e6 oncogene protein, endostatin, epidermal growth factor receptor, epithelial cell adhesion molecule, Erk2, Estrogen receptor a, Eukaryotic translation initiation factor 4G, factor d, Factor IX, factor ixa, factor viia, factor x, factor xa, fibrinogen, fibronectin, fibronectin binding protein a, fibronectin binding protein b, fractalkine, G-protein-coupled receptor kinase 2, G6-9 anti-DNA autoantibody, ga733-1, ghrelin, glyceraldehyde-derived pyridinium, glycine receptor (glyr), Glycoprotein VI, gonadotropin, gonadotropin-releasing hormone I, gper neurotensin, HA Binding Domain of Human CD44, HbA1c, heat shock factor, hemoglobin, heptaocyte growth factor, histone H4, hmg-1, hnspA2 human nonpancreatic secretory phospholipase A2, hsp90-binding immunophilin, human acetylcholinesterase, Human Cardiac Troponin I, human CD73, human complement 5, human dicer, human epidermal growth factor receptor 2, Human epidermal growth factor receptor-3 (HER3), human erythropoietin-a (rHuEPO-a), human gp73, human growth hormone, human heat shock factors 1, human heat shock factors 2, Human Hepatocyte Growth Factor, human interleukin-8, human matrix metalloprotease 9 (hMMP-human neutrophil elastase, human periostin, human plasma, human Platelet-Derived Growth Factor chain B, Human Pro-Urokinase, human Rad51, human rad51, human RNase H1, human thyroid stimulating hormone, Human tPA, human transferrin receptor, human β2-microglobulin, immunoglobulin e, immunoglobulin g, insulin, insulin receptor, integrin α4, integrin αVβ3, interferon γ, interferon γ induced protein 10, interferon-inducible t-cell α chemoattractant, interleukin-10ra, interleukin-12, interleukin-16, Interleukin-17, interleukin-23, interleukin-6, interleukin-6 receptor, interleukin-8, k ras-derived farnesylated peptide, kallikrein-related peptidase 6 (klk6), keratinocyte growth factor, L-selectin, L7Ae protein, leptin, leptin r, lipocalin-2, Lysozyme, mAb198, macrophage migration inhibitory factor, MAGE-A3111-125, matn2, Mek1, metastatic enzyme heparanase (HPSE1), MetJ (methionine repressor), migraine-associated calcitonin gene-related peptide, mitochondrial GTPase NOA1, mitogen-activated kinase, monoclonal antibody 83-7, mouse CCL1, mouse glycoprotein 2, mouse monoclonal antibody ma20, mouse vcam-1, MRCKα-KD, MUC1, MUC16 ca125 ovarian cancer cell marker, murine c-type receptor dec205, murine Interleukin-10 (IL-10), murine myelin, mutant KRAS (G12V), MutS, myelin basic protein, N-methyl D-aspartate (NMDA) class of ionotropic glutamate receptors, negative elongation factor e, neuropeptide Y, Neurotrophin Receptor, neutrophil elastase, NF-IL6, nogo-66 receptor, nts-1, nuclear factor of activated T cells, nuclear factor κβ, nucleolin, nucleophosmin, oncogene tiam1, Oncogenic Protein Shp2, osteopontin, osteoprotegerin, ovarian cancer biomarker HE4, OX40, p-cadherin, P-selectin, P2X2 receptors, p43, p50, p53R175H, PAI-1, PAK1, pancreatic adenocarcinoma up-regulated factor, PDGF-BB, Pepocin, Peroxisome proliferator-activated receptor δ, phospholamban, Plasminogen Activator Inhibitor-1, plg, prostate specific antigen, prostatic acid phosphatase, protein kinase C-d, PTFase, Pulmonary Surfactant Protein A, quinoprotein glucose dehydrogenase (PQQGDH), Ras, Ras-binding domain of Raft, recombinant human apc, recombinant human growth hormone (rhgh), rela (p65), Retinol binding protein 4 (RBP4), rev nuclear export signal, Rho, RIG-I, RNA binding domain, ma polymerase σ, RNase, RUNX1, schlerostin, se-selectin, Sec7 domain of cytohesin 1, SeIB (elongation factor for selenocystein incorporation), serine protease urokinase-type plasminogen activator, sphingomyelinase, SRP19, substance P, t-cell 4-1bb, tenascin C, TFIIIA, TGF-b Receptor II, thrombin, Thrombospondin 2, thyroid transcription factor 1, toll-like receptor 2, transferrin, transforming growth factor receptor b2, transforming growth factor-b1, transforming growth factor-β type III receptor, TrkB, trypsin, tumor necrosis factor receptor super family member 9, tyrosine kinase RETC634Y, tyrosine phosphatase 1b, Tyrosine phosphatase SHP2, UBLCP1, unglycosylated epidermal growth factor receptor viii ectodomain, urokinase plasminogen activator, vascular endothelial growth factor, vascular endothelial growth factor 165, vasopressin, vimentin, von willebrand factor, von willebrand factor al-domain, ZAP, Zinc Finger Proteins, α-fetoprotein, α-synuclein, β-arrestin 2, β-catenin, β-Conglutin, and β-site amyloid precursor protein cleaving enzyme1 (bace1).

Additional examples of proteins for which DNA aptamers have been developed include ara h 1 allergen, asp f 1 allergen, bacterial RNA polymerase, Caenorhabditis elegans bcl-2 homolog ced-9, CFP, Concanavalin A, cry j 2 allergen, E. coli core RNA polymerase, electric eel acetylcholinesterase, eotaxin, erf1, Escherichia coli methionine repressor, Escherichia coli release factor 1, f(ab′)2 fragments of saxitoxin (stx) antibodies, GFP, heterogenous ribonucleoprotein I (hnrnp I), horse radish peroxidase, i-scei endonuclease, initiation factor 4a, innexin 2, inosine monophosphate dehydrogenase, lup an 1, mitochondrial processing peptidase, okadaic acid monoclonal antibody, peptidoglycan, sA from Thermus aquaticus, streptavidin, subtilisin (protease), systemin, T4 DNA pol, t7 ma polymerase, taq dna pol, tbp (tats box protein), Tet Repressor, tfiib, TIMP1, tobacco protoplast protein, yeast RNA polymerase II (Pol yeast TATA-binding protein, yeast TFIIB, and YFP.

In some embodiments, the DNA aptamer binds a cell target. Examples of non-cancerous mammalian cells for which DNA aptamers have been developed include 3T3-L1 adipocytes, Adult mesenchymal stem cells, BJAB cells expressing c-kit, C666-1, CD81 T-cells, Cell internalization, Differentiated PC12 cells, cho-k1 cells expressing human endothelin type b receptor (etbr), HEK-293, Transformed tonsillar epithelial cells, Human jaw periosteal cells, Human platelets, Inflamed endothelial cells, Malaria-infected RBCs, Mature white adipocytes, MCF-10AT1, MiaPaCa-2 secretome, Mitochondria, NP69, Osteoblasts, PC-3, PC:cholesterol liposomes, Rabies virus-infected live cells, RSV transformed SHE cells, and Transformed tonsillar epithelial cells.

Examples of pathogenic microorganisms for which DNA aptamers have been developed include African Trypanosomes, Alicyclobacillus spores, Anthrax spores, Bacillus spores, Bacillus thuringiensis, Campylobacter jejuni, Cryptosporidium parvum, Escherichia coli DH5a, Escherichia coli K12, Escherichia coli NSM59, Escherichia coli O111:134, Escherichia coli O157:H7, Francisella tularensis, Lactobacillus acidophilus, Leishmania major promastigotes, Listeria monocytogenes, Mycobacterium tuberculosis, Porphyromonas gingivalis, Proteus mirabilis, Pseudomonas aeruginosa, Salmonella choleraesuis, Salmonella enteritidis, Salmonella O8, Salmonella paratyphi A, Salmonella typhimurium, Shigella dysenteriae, Staphylococcus aureus, Streptococcus mutans, Streptococcus oralis, Streptococcus pyogenes, Streptococcus sanguis, Treponema denticola, Trypanosoma cruzi, Tuberculosis, Vibrio alginolyticus, and Vibrio parahemolyticus.

Examples of cancer cells for which DNA aptamers have been developed include Acute myeloid leukemia (AML) cells, Adenocarcinoma, BG-1 ovarian cancer cells, Brain Tumor-Initiating Cells, Breast cancer, Burkitt lymphoma cells, Cancer stem cells, Colon cancer cell SW620, Colorectal cancer cell line DLD-1, CT26 intrahepatic tumor, Epithelial cancer cells, Gastric cancer cell-line HGC-27, Gefitinib-resistant H1975 lung cancer cells, Glioblastoma multiforme, Hepatocellular carcinoma, HER2 positive cell line, HPV-transformed cervical cancer cells, Human breast cancer MDA-MB-231, Human cholangiocarcinoma QBC-939 cells, Human gastric carcinoma AGS, Human glioblastoma multiforme cells overexpressing epidermal growth factor receptor variant III, Human hepatocarcinoma, Human pancreatic ductal adenocarcinoma, Human U87MG glioma cells, Leukemia cells, Liver cancer, MCF-10AT1, MDA-MB-231 breast cancer, Metastatic colorectal cancer, Metastatic hepatocellular carcinoma cells, MS03 cancer line, Ovarian cancer cell TOV-21G, Ovarian serous adenocarcinoma cell CAOV-3, Pancreatic cancer cells, Primary Cultured Tumor Endothelial Cells, Primary human chronic lymphocytic leukemia, Ramos cells, Rat brain tumor microvessels, Small-cell lung cancer cells, SMMC-7721 liver carcinoma cells, and Vaccinia virus-infected lung cancer A549 cells.

Examples of nucleic acid targets for which DNA aptamers have been developed include Bacillus subtilis RNase P P5.1 stem-loop element, DNA/RNA motifs, HCV IRES, HIV-1 TAR element, PCA3 RNA, Target A-site 16S rRNA, and Yeast phenylalanine tRNA.

Examples of viral targets for which DNA aptamers have been developed include apple stem pitting virus, Arbovirus, Bovine viral diarrhea virus type 1, Fish Pathogen Viral Hemorrhagic Septicemia Virus, Herpes simplex virus type 2, Hirame rhabdovirus, HIV-1 subtype C envelope pseudovirus, Human cytomegalovirus, Human Norovirus, Influenza A/H1N1, Influenza A/H3N2, Influenza A/H5N1, Influenza B/Tokio/53/99, Influenza B/05/99, Singapore grouper iridovirus, Soft-shelled turtle iridovirus, Tobacco Necrosis Virus, Vaccinia virus, and Vesicular stomatitis virus (VSV).

In some embodiments, the DNA aptamer binds a small molecule target. Examples of fluorophores for which DNA aptamers have been developed include aniline-based quencher, Cibacron Blue 3GA, Cy3, DFHBI, Dihydropyrene photo-switch compound, Dimethylindole Red, DMABI, DMHBI, Fluoroscein, Hoechst derivative 7, Reactive Blue 4, Reactive Brown 10, Reactive Green 19, Reactive Red 120, Reactive Yellow 86, Sulforhodamine, Tetramethylrhodamine, and Thiazole orange.

Examples of pharmaceuticals for which DNA aptamers have been developed include (1-3)-b-D-glucans, 2-anilinophenylacetic acid, Acetamiprid, Aminoglycoside antibiotic, Chloramphenicol, Citrulline, Codeine, Cyclosporin A, Danofloxacin, Daunomycin, Diclofenac, Digoxin, Gentamicin, Globo H, Glutathione, Hematoporphyrin, Heteroaryldihydropyrimidine, Ibuprofen, Kanamycin, Lividomycin, Lysergamine, Metergoline, Moenomycin A, Neomycin, Paromomycin, Poly-γ-D-glutamic acid (g-PDGA), R-Thalidomide, Small Ergot Alkaloids, Spectinomycin, Streptomycin, Sulfadimethoxine, Tetracycline, Theophylline, and Tobramycin.

Examples of toxins and environmental hazard small molecules for which DNA aptamers have been developed include 2,4,6-trichloroaniline (TCA), Abrin toxin, Acetamiprid, Aflatoxin B1, Aflatoxin M1, Bisphenol A, Brevetoxin, Carcinogenic aromatic amines, Chinese Horseshoe Crab endotoxin, cylindrospermopsin, Digoxin, Dinitroaniline, Ethanolamine, Fumonisin B1, Isocarbophos, Lipopolysaccharide, Neurotoxin anatoxin-a, Ochratoxin A, Okadaic acid, Omethoate, P-aminophenylpinacolylmethylphosphonate, Pentachlorophenol, Phorate, Polychlorinated biphenyls, Profenofos, Staphylococcus aureus enterotoxin A, Trinitrotoluene, and zearalenone.

Examples of amino acids and peptides for which DNA aptamers have been developed include Arginine, Citrulline, Glutamic acid, Glutathione, Histidine, His Tag 6×, Isoleucine, L-arginine, L-tryptophan, P-amino phenylalanine, P-amino phenylalanine, Peptide: Asp-Gly-Ile, Peptide: Gly-Glu-Leu, Peptide: His-Phe, Peptide: Leu-Ala-Ser, Peptide: Lys-Ala-Ile, Phenylalanine, S-adenosyl methionine, S-Adenosylhomocysteine, Tachykinin substance P, Tryptophan, Tyrosine, and Valine.

Examples of metals for which DNA aptamers have been developed include Cadmium, Nickel, Palladium ion, Uranyl ion, and Zinc.

Examples of biologics and signaling molecules for which DNA aptamers have been developed include Acetylcholine, Biotin, cAMP, Cellulose, Cholic acid, CoA, Cortisol, Cyanocobalin (vitamin B12), Dehydroisoandro sterone-3-sulfate, Deoxy-corticosterone-21 glucoside, Deoxycholic acid sodium salt, Dopamine, Flavin, Fructose, Galactose, Glucagon, Glucose, Hemin, Hormone Abscisic Acid, N-acetylneuraminic acid, n-glycolylneuraminic acid (neu5gc), Nicotinamide, R-Thalidomide, Sialyl Lewis X, Sialyllactose, Sphingolipid S1 P, Sphingosylphosphorylcholine, Steroid, Thiamine pyrophosphate, Thyroxine hormone, thyroxine hormone, Urea, Vasopressin, Vitamin D, Zeatin, and β-estradiol.

Examples of nucleosides and nucleotides for which DNA aptamers have been developed include 8-hydroxy-2′-deoxyguanosine, Adenosine, ADP, AMP, ATP, GMP, GTP, Guanine, and Xanthine.

Examples of synthetic small molecules for which DNA aptamers have been developed include 4-chloroaniline (4-CA), Biotin pyridocarboxamide derivative, Bis-boronic acid receptor, L-tyrosinamide, Methylphosphoic acid, N-methyl mesoporphyrin IX, P-nitrobenzene sulfonyl, and Tartrate.

Additional protein targets can

Collagen Crosslinking

The crosslinking reaction may be carried out by combining collagen and a DNA aptamer as disclosed herein at relative amounts effective to produce collagen fibers. 8 to 30% mass fraction of ssDNA in solution.

The crosslinking reaction may be carried out at a temperature according to the judgment of those of skill in the art. In certain embodiments, the crosslinking reaction is carried out at about 0-50° C., about 20-50° C., about 20-45° C., about 20-40° C., about 20-35° C., or about 20-30° C. In other embodiments, the crosslinking reaction is carried out at about 0° C., about 5° C., about 10° C., about 15° C., about 20° C., about 25° C., about 30° C., about 35° C., about 40° C., about 45° C., or about 50° C. In particular embodiments, the crosslinking reaction is carried out at about 20-40° C.

The crosslinking reaction may be carried out at a pH according to the judgment of those of skill in the art. For example, it is well-known in the art that crosslinking agents are effective at crosslinking at a particular pH or ranges of pH. In certain embodiments, the crosslinking reaction is carried out at a pH of about 6-12, about 7-12, about 7-11, about 7-10, or about 7.2-10. In other embodiments, the crosslinking reaction is carried out at a pH of about 6, about 7, about 7.2, about 9, about 10, about 11, or about 12.

The crosslinking reaction may be carried out for a period of time according to the judgment of those of skill in the art. In certain embodiments, the crosslinking reaction is carried out for about 1 minute, about 30 minutes, about 1 hour, about 2 hours, about 3 hours, about 4 hours, about 5 hours, about 10 hours, about 16 hours, about 20 hours, about 24 hours, about 40 hours, about 48 hours, or about 72 hours.

The concentration of DNA aptamer used in the crosslinking reaction may be a concentration according to the judgment of those of skill in the art. In certain embodiments, the concentration of the DNA aptamer is about 0.00005-0.0005%, about 0.0001-0.001%, or about 0.00025-0.0025%.

In embodiments, elastin can be combined with collagen. In embodiments, elastin can be added in a ratio of about 1:1 to about 1:2 elastin:collagen.

Collagens

The collagen starting material used for producing crosslinked collagen material of the present invention can be a collagen or collagens of any type. In certain embodiments, the crosslinked collagen material of the present invention is produced from a collagen starting material comprising a fibril forming collagen. Fibril forming collagens include type I, type II, type III, type V, and type XI collagens. In other embodiments, the crosslinked of the present invention is produced from a collagen starting material comprising a fibril associated collagen. Fibril associated collagens include type IX, type XII, type XIV, type XVI, type XIX, and type XXI collagens. In other embodiments, the crosslinked collagen material of the present invention is produced from a collagen starting material comprising a sheet forming collagen. Sheet forming collagens include type IV, type VIII, and type X collagens. In yet other embodiments, the crosslinked collagen material of the present invention is produced from a collagen starting material comprising a beaded filament collagen or an anchoring fibril collagen. Beaded filament collagens and anchoring filament collagens include type VI collagen and type VII collagen, respectively. Other collagen types useful in the present methods include type XIII, type XV, type XVII, type XVIII, type XX, type XXII, type XXIII, type XXIV, type XXV, type XXVI, type XXVII, and type XXVIII collagen. (See Haralson and Hassell, Extracellular Matrix, A Practical Approach, 8-11, Oxford University Press, 1995, the contents of which is hereby incorporated by reference in its entirety.) In a particular embodiment, a fibril forming collagen (i.e., type I, type II, type III, type V, or type XI collagen) is the collagen starting material used to produce crosslinked collagen according to the methods of the present invention.

In one embodiment, the collagen starting material useful for producing crosslinked collagen material is recombinant collagen. In another embodiment, the collagen starting material useful for producing crosslinked collagen material is recombinant human collagen. The use of any single type of recombinant collagen (e.g., recombinant type I collagen, recombinant type II collagen, recombinant type III collagen, etc.) or any mixture of more than one type of recombinant collagen (e.g., a mixture of recombinant type I collagen and recombinant type III collagen) as the collagen starting material for producing a crosslinked collagen material is specifically contemplated by the present invention. Recombinant collagens and methods of their production have been described in, e.g., International Publication Nos. WO 2006/052451 and WO 1993/007889, each of which is hereby incorporated by reference in its entirety.

Production of other collagens suitable for use in the present compositions and methods can be specifically engineered using molecular biology techniques known to one of skill in the art. Such collagens can be modified by, e.g., an alteration in the polypeptide coding sequence, including deletion, substitutions, insertions, etc., to increase resistance to degradation. For example, recombinant collagens with alterations in the amino acid sequence at specific protease cleavage sites can be produced. Accordingly, in one embodiment, the present invention provides novel compositions comprising collagen, wherein the collagen is a recombinant Type III collagen.

The methods of the present invention are useful for producing crosslinked collagen materials using recombinant collagen (e.g., recombinant human collagen) as the collagen starting material. Unlike naturally-derived collagens, recombinant collagens lack intermolecular and intramolecular crosslinks that, if present, help stabilize the collagen material (including collagen fibrils) under conditions suitable for various crosslinking reactions, including, for example, basic pH conditions (e.g., pH ≥8) or increased temperature (e.g., temperature ≥40° C.). Under such conditions, recombinant collagens and, in particular, recombinant collagen fibrils made from recombinant collagens, are unstable, resulting in fibril dissolution and triple helix melting.

In aspects of the present disclosure, collagens can be present in a hydrogel composition.

Crosslinked Collagen Materials

The present invention provides crosslinked collagen materials. In some embodiments, the invention provides crosslinked recombinant collagen suitable for implantation into a human or animal body. Such a crosslinked recombinant collagen implant is suitable for medical or cosmetic use. Typically, crosslinked recombinant collagen according to the invention is implanted or injected into various regions of the skin or dermis, depending on the particular application or cosmetic procedure, including dermal, intradermal, and subcutaneous injection or implantation. The crosslinked collagen materials of the present invention can also be injected or implanted superficially, such as, for example, within the papillary layer of the dermis, or can be injected or implanted within the reticular layer of the dermis. Materials for injection or implantation into the skin, in particular for cosmetic benefit, are often referred to in the art as “dermal fillers”. Accordingly, in one embodiment, a dermal filler, typically a cosmetic dermal filler, comprising crosslinked recombinant collagen according to the invention is provided.

The crosslinked collagen materials of the present invention may be used to produce implantable collagen compositions. Production of implantable collagen compositions has been described in, e.g., International Publication No. WO 2006/052451, the contents of which is hereby incorporated by reference herein in its entirety. In certain embodiments, the present invention provides implantable collagen compositions, comprising at least one crosslinked collagen material. The crosslinked collagen material can be any crosslinked collagen of the invention, for instance crosslinked “fibril forming” collagen materials prepared by one of the methods described herein. In one aspect, the implantable collagen composition comprises crosslinked recombinant type III collagen material.

The crosslinked collagen materials of the present invention can be formulated or used at any concentration useful to those of skill in the art. In certain embodiments, the formulations of the materials of the invention comprise 0.03-0.3 mg/ml, 1-10 mg/ml.

It is understood that the compositions of the present invention can include additional components suitable to the particular formulation. For example, in certain embodiments, the implantable compositions of the present invention are intended for injection and are formulated in aqueous solutions. The compositions can be formulated to include pharmaceutically acceptable carriers and excipients. Such carriers and excipients are well-known in the art and can include, e.g., water, phosphate buffered saline (PBS) solutions, various solvents, and salts, etc., for example, physiologically compatible buffers including physiological saline buffers such as Hanks solution and Ringer's solution.

The amount of crosslinked collagen material appropriately included in a particular formulation is determined as standard in the art for such formulations, and is dictated by the intended use. In certain embodiments, the present invention provides implantable compositions comprising crosslinked collagen material wherein the collagen material is in aqueous solution at a concentration between about 0.03 to about 10 mg/ml.

Magnetoelectric Composite Materials

Described herein are magnetoelectric composite materials, in addition to compositions, methods, and kits comprising such. Magnetoelectric composite materials represent an aspect of improving the function of collagen materials as described herein, in particular in conjunction with the application of a magnetic field, and electric field, or both.

In embodiments, magnetoelectric composite materials comprise electrospun fibers. In embodiments, magnetoelectric composite materials comprise nanowires, nanoparticles, and thin films. Magnetoelectric composite materials can have morphologies that are randomly dispersed, core-shell, or Janus.

Magnetoelectric composite materials as described herein can be comprised of piezoelectric (e.g. BaTiO3, PbZrTiO3, BiFeO3, among others) and magnetostrictrive (E.g., CoFe2O4, Fe3O4, Galfenol, Terfenol, among others). In embodiments, magnetoelectric composite materials as described herein comprise BaTiO₃ and CoFe₂O₄. In embodiments, magnetoelectric composite materials as described herein consist of BaTiO₃ and CoFe₂O₄.

In embodiments, magnetoelectric composite materials as described herein are fibers with a length of about 1 μm to about 100 μm. In embodiments, magnetoelectric composite materials as described herein have a diameter of about 100 to about 1000 nm.

In an embodiment, magnetoelectric composite materials as described herein have a composition of ˜43 wt. % of pure cobalt ferrite fibers. The composition range can span 2-98 wt. % cobalt ferrite.

Methods of Using Crosslinked Collagen Materials and Magnetoelectric Composites

The crosslinked collagen materials provided herein can be used in any method known or contemplated by those skilled in the art in combination with magnetoelectric composite materials as described herein. In particular, the present crosslinked collagen materials can be used in any of the numerous medical and cosmetic applications, including tissue augmentation procedures, in which collagen is currently used. The present crosslinked collagen materials are suitable for use in tissue augmentation procedures. Use of the present crosslinked collagen materials in cosmetic as well as in medical procedures is specifically provided.

In one aspect, the present invention provides implantable compositions containing crosslinked collagen materials suitable for use in soft tissue augmentation procedures. The present compositions can be implanted or injected into various regions of the skin or dermis, depending on the particular application or cosmetic procedure, including dermal, intradermal, and subcutaneous injection or implantation. The crosslinked collagen materials of the present invention can also be injected or implanted superficially, such as, for example, within the papillary layer of the dermis, or can be injected or implanted within the reticular layer of the dermis.

In addition to soft tissue augmentation, use of the crosslinked collagen materials for hard tissue augmentation is provided by the present invention. The present crosslinked collagen materials are useful in various hard tissue augmentation applications, including, for example, as a bone-void filler, dental implant, etc.

Cosmetic uses of the crosslinked collagen materials of the present invention include treatment of fine lines, such as fine superficial facial lines, wrinkles, and scars, as well as treatment of pronounced lines, wrinkles, and scars. In some aspects, the crosslinked collagen materials of the present invention are used for other cosmetic uses, including treatment for or reducing transverse forehead lines, glabellar frown lines, nasolabial fold, vermilion border, periorbital lines, vertical lip lines, oral commissure, etc., as well as defining the lip border. The crosslinked collagen materials of the present invention are also useful for correcting contour deformities and distensible acne scars, or for treating other tissue defects, such as, for example, atrophy from disease or trauma or surgically-induced irregularities.

In certain embodiments, the crosslinked collagen materials of the present invention are used for surgical procedures involving tissue augmentation, tissue repair, or drug delivery. In some aspects, the crosslinked collagen materials are used for tissue augmentation in conditions such as urinary incontinence, vasicoureteral reflux, and gastroesophageal reflux. For example, crosslinked collagen materials of the present invention may be used to add tissue bulk to sphincters, such as a gastric or urinary sphincter, to provide proper closure and control. In instances of urinary incontinence, such as stress incontinence in women or incontinence following a prostatectomy in men, the crosslinked collagen materials of the invention may be provided to further compress the urethra to assist the sphincter muscle in closing, thus avoiding leakage of urine from the bladder.

Similarly, gastroesophageal reflux disease (GERD, also known as peptic esophagitis and reflux esophagitis) is a disorder that affects the lower esophageal sphincter, the muscle connecting the esophagus with the stomach. GERD occurs when the lower esophageal sphincter is incompetent, weak, or relaxes inappropriately, allowing stomach contents to flow up into the esophagus (i.e., reflux). Malfunction of the lower esophageal sphincter muscles, such as that resulting from muscle tonal loss, can lead to incomplete closure of the lower esophageal sphincter, causing back up of acid and other contents from the stomach into the esophagus. Poor response to dietary modification or medical treatment may require surgery to correct the dysfunction. In one embodiment, crosslinked collagen materials of the present invention are used in such procedures and, for example, are injected into the area of the esophageal sphincter to provide bulk to the lower esophageal sphincter.

In other embodiments, the crosslinked collagen materials of the invention are used to fill or block voids and lumens within the body. Such voids may include, but are not limited to, various lesions, fissures, diverticulae, cysts, fistulae, aneurysms, or other undesirable voids that may exist within the body; and lumens may include, but are not limited to, arteries, veins, intestines, Fallopian tubes, and trachea. For example, an effective amount of the present material may be administered into the lumen or void to provide partial or complete closure, or to facilitate repair of damaged tissue.

In other aspects, tissue repair is achieved by providing the crosslinked collagen material of the present invention to an area of tissue that has been diseased, wounded, or removed. In some embodiments, crosslinked collagen materials of the invention are used to fill in and/or smooth out soft tissue defects such as pockmarks or scars. In such cases, a formulation of the present invention is injected beneath the imperfection. The improved persistence of the present crosslinked collagen materials would be beneficial, e.g., by reducing the number and frequency of treatments required to obtain a satisfactorily result. In certain embodiments, the crosslinked collagen materials are used for intracordal injections of the larynx, thus changing the shape of this soft tissue mass and facilitating vocal function. Such use is specifically provided for the treatment of unilateral vocal cord paralysis. Further, the present invention provides use of the crosslinked collagen materials in mammary implants, or to correct congenital anomalies, acquired defects, or cosmetic defects.

The present crosslinked collagen materials can also be used in various surgical or other procedures for remodeling or restructuring of various external or internal features, e.g., plastic surgery for corrective or cosmetic means, etc.

In any of the embodiments described above, the present crosslinked collagen materials may be used for drug delivery, for example, to deliver drugs to an injection site. The drugs can be delivered in a sustained manner from an in vivo depot formed by the crosslinked collagen upon injection of an implantable composition of the present invention. Drugs delivered in this manner may thus enhance tissue repair, and could provide additional therapeutic benefit.

In additional embodiments, the invention further contemplates incorporation of cells into the crosslinked collagen materials to provide a means for delivering cells to repopulate a damaged or diseased tissue or to provide products synthesized by the cells to the tissues surrounding the injection site.

In any of the embodiments described above, the crosslinked collagen materials of the present invention may be delivered or administered by any suitable method known or contemplated by those of skill in the art. The invention specifically contemplates delivery by injection, e.g., using a syringe. In some embodiments, the crosslinked collagen materials may additionally contain a biocompatible fluid that functions as a lubricant to improve the injectability of the formulation. The crosslinked collagen materials of the invention can be introduced into the tissue site by injection, including, e.g., intradermal, subdermal, or subcutaneous injection.

Methods of Tissue Culture

Described herein are methods of tissue culture comprising magnetoelectric composite materials as described herein in addition to collagen materials as described herein. Methods as described herein can further comprise the use of nucleic acids.

Methods of tissue culture as described herein can comprise culturing one or more cells according to methods as known in the art in the presence of magnetoelectric composite materials and collagen materials as described herein. Methods can further comprise culture one or more cells with materials as described herein in the presence of nucleic acids.

According to methods of tissue culture according to the present disclosure, magnetic and/or electric fields can be applied for a period of time during culture.

Magnetic fields can be applied at a strength of about 0 to about 5 mT.

Electric fields can be generated and applied at a strength of about 0 to about 500 mV/mm.

Electric and/or magnetic fields can be applied for a period of time of about 0 min to days. Such fields can be applied continuously, or then can be applied sporadically and “pulsed” periodically over a period of time.

Cells as described herein can be any mammalian cells. In embodiments, cells as described herein can be neuronal progenitor cells. In embodiments, cells as described herein can be neuronal cells. In embodiments, cells as described herein can be epithelial cells. In embodiments, cells as described herein can be osteoclasts or osteoblasts. Cells as described herein can be mammalian cells, for example human, rat, and/or mouse cells.

Kits Comprising Crosslinked Collagen Materials

One embodiment of the present invention provides kits comprising the crosslinked collagen materials of the invention. For example, the present invention provides kits for augmenting or replacing tissue of a mammal. The kits comprise one or more crosslinked collagen materials of the present invention in a package for distribution to a practitioner of skill in the art. The kits can comprise a label or labeling with instructions on using the crosslinked collagen material for augmenting or replacing tissue of a mammal according to the methods of the invention. In certain embodiments, the kits can comprise components useful for carrying out the methods such as means for administering a crosslinked collagen material such as one or more syringes, canulas, catheters, needles, etc. In certain embodiments, the kits can comprise components useful for the safe disposal of means for administering the crosslinked collagen material (e.g. a ‘sharps’ container for used syringes). In certain embodiments, the kits can comprise crosslinked collagen material in pre-filled syringes, unit-dose or unit-of-use packages.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill CO.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

Embodiments of kits of the present disclosure can comprise magnetoelectric composite materials in addition to any other combination of materials (collagen materials, cross-linked collagen materials, nucleic acids, for example) as described herein.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Effect of Premixing Aptamer and Collagen Versus Pre-Conjugation of Aptamer Followed by Addition of Collagen

The effect of premixing aptamer and collagen versus pre-conjugate of aptamer followed by addition of collagen was examined.

Materials and Methods

Aptamer Sequences

scrambled=5AmMC6/TAAAACGCGCTTAAGCTGGTGTTACTCGAGCGGTCTTCTATTGAAATAATT TCTGAAGGCACACGACATATGATCTTCAG (SEQ ID NO:1). 5AmMC6 specifies a terminal amino group with 6 carbon spacer was conjugated to the 5′ end of the oligonucleotide sequence.

Experimental Conditions

Mixtures of scrambled aptamer (1 μM) were mixed with rat tail type I collagen (0.3 mg/mL). Mixtures were at 10, 30, 50% volume fraction collagen. Mixtures were incubated at room temperature for 24 hours prior to surface conjugation. Solutions were used to conjugate to surface with 20 μM sulfo-SANPAH and incubated at room temperature for 24 hours. Surfaces were also conjugated with 1 and 2 μM solutions of scrambled aptamer and 20 μM sulfo-SANPAH and then incubated with 0.3 mg/mL and 0.6 mg/mL solutions of rat tail type I collagen.

Results

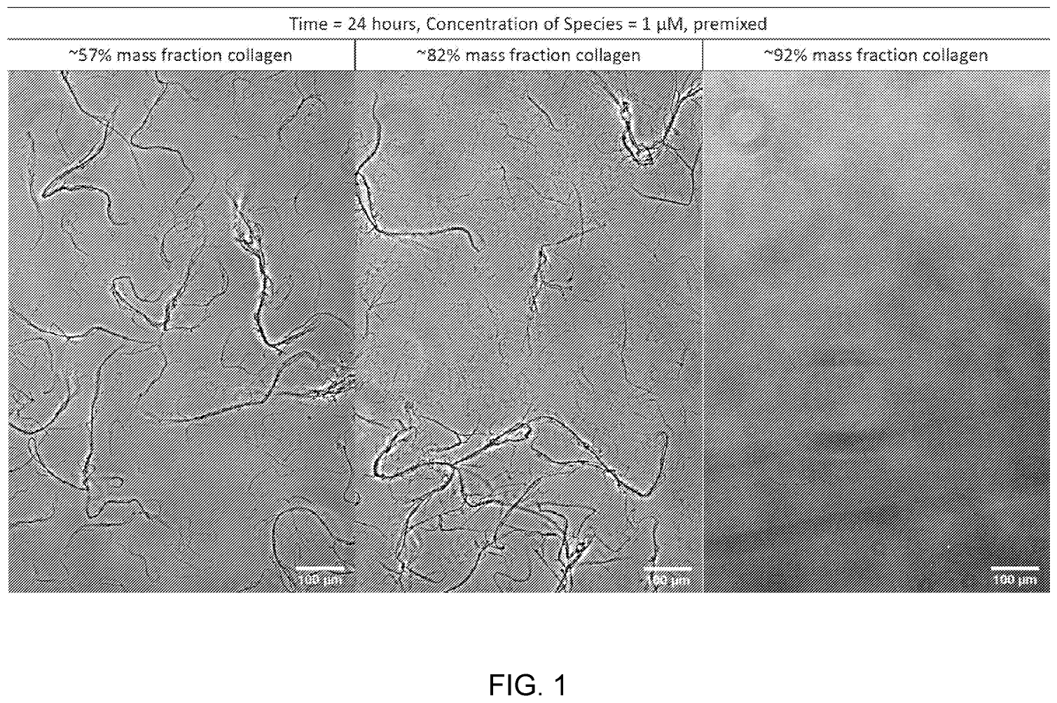

As show in FIG. 1, with premixed solutions, large fibers formed at 10 and 30% volume fraction collagen. At 30% volume fraction collagen surfaces and fibers appeared fuzzy for all aptamers. At 50% volume fraction there were no fibers.

With pre-conjugation with aptamer, there was no discernable fiber formation at all treatments. Surface wetting character observed to be changed from that of only an aptamer conjugated surface, which indicates collagen has adsorbed to the surface. The aptamer needs to have free mobility for fiber formation.

### Example 2: Aptamer-Collagen Complex Kinetics Measured by Turbidity

Materials and Methods

Experimental Conditions

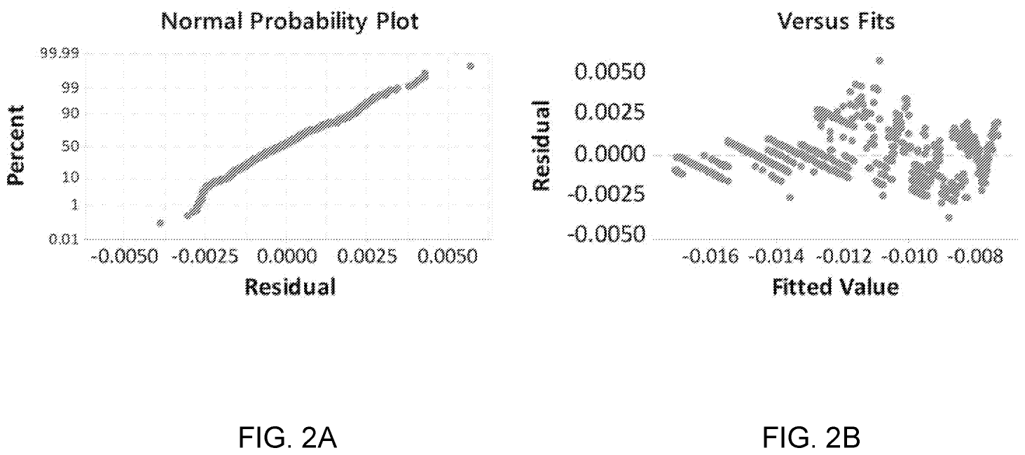

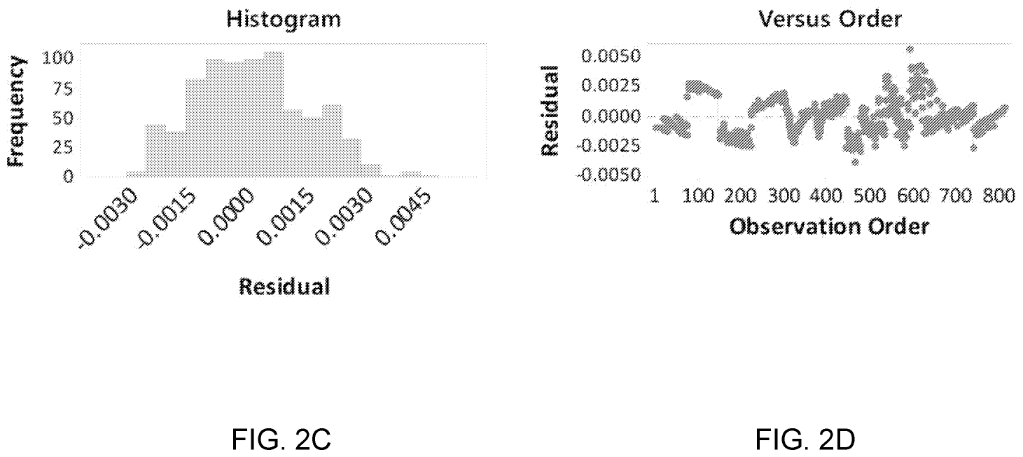

In 96 well plate, mixtures of scrambled aptamer (1 μM) were mixed with rat tail type I collagen (0.3 mg/mL). Mixtures were at 10% increments from 0% to 100% volume fraction collagen with 2 replicates. Turbidity measured over time immediately following mixing for six hours at 5 minute increments at 400 nm using a BioTek plate reader in absorbance mode. Data was fit with 3 rd order interactive regression model with factors of time, collagen fraction, and aptamer type (FIGS. 2A to 2D).

Results

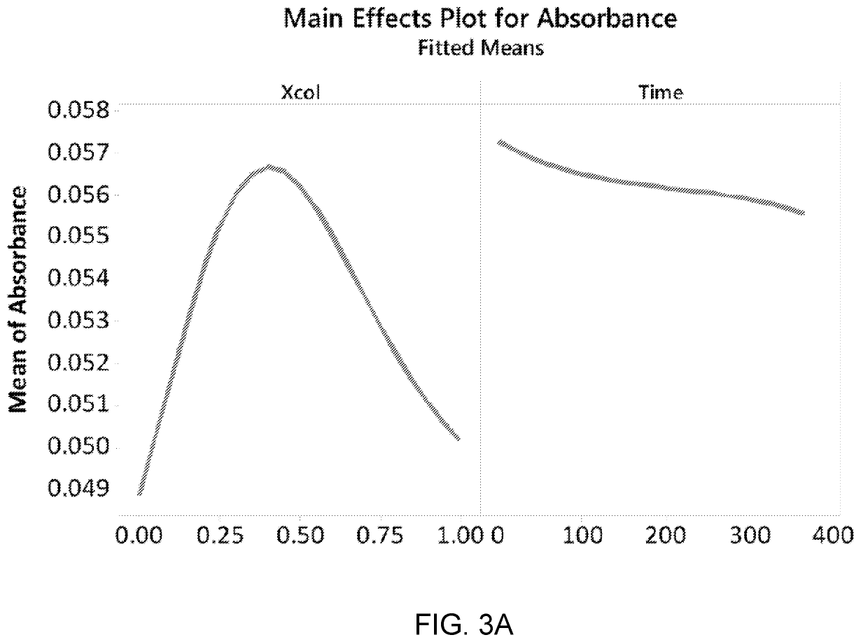

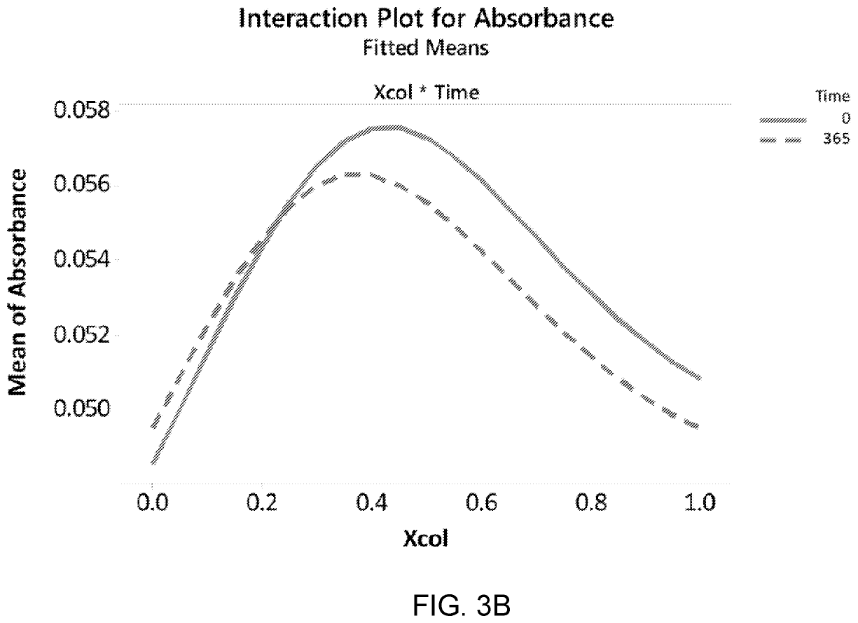

Turbidity showed little change with time (FIG. 3A). Turbidity showed maximum at volume fraction collagen (FIG. 3B).

### Example 3: Confirmation of DNA Present in Self-Assembled Collagen Fibers

Materials and Methods

Experimental Conditions

In 96 well plate, mixtures of scrambled aptamer (1 μM) were mixed with rat tail type I collagen (0.3 mg/mL). Mixtures were at 10% increments from 0% to 100% volume fraction collagen with 2 replicates. Mixtures were incubated at room temperature for 24 hours. 10 μL aliquots of 30 and 70% volume fraction collagen were taken and placed on slides. They were then stained with ethidium bromide homodimer.

Results

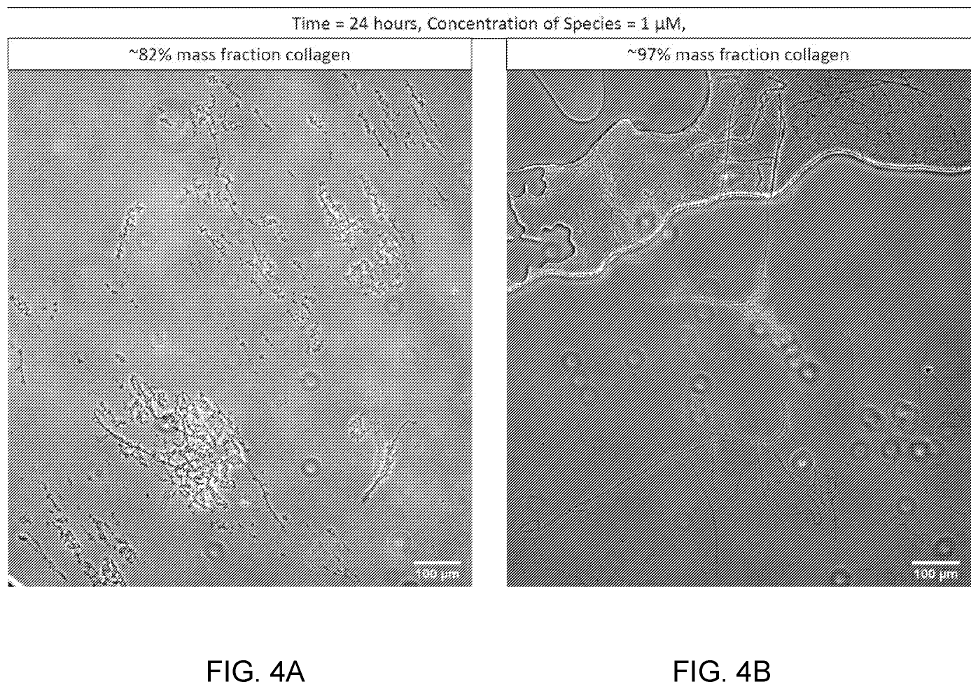

Fibers were stained for DNA at high and low concentrations of collagen. Structurally fibers looked fuzzy at 30% collagen and more whispy/defined at 70% collagen (FIGS. 4A and 4B).

### Example 4: Aptamer-Collagen Binding Measurement

Materials and Methods

Experimental Conditions

In 96 well plate, mixtures of scrambled aptamer (1 μM) were mixed with rat tail type I collagen (0.3 mg/mL). Mixtures were at 25% increments from 0% to 100% volume fraction collagen with 1 replicate. Solutions were incubated at room temperature for 2 hours and then SYBR Safe DNA stain was added in a 2:1 ratio to the aptamer-collagen solutions and incubated for 30 minutes in the dark at room temperature. The solutions were centrifuged at 2000 g for 5 minutes and the supernatant was added to a 96 well plate giving 2 repeats per solution. Fluorescent intensity was measured by exciting at 488 nm and reading emission at 520 nm.

Results

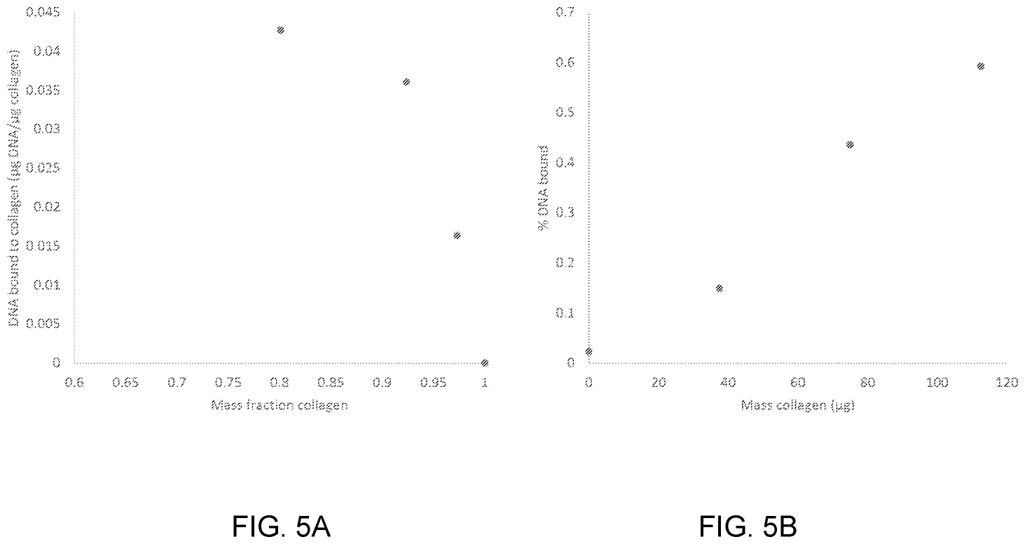

Standard curves were linear. The percent DNA bound increased with increased amount of collagen in solution (FIG. 5B). The amount of DNA bound to a given amount of collagen decreased with increased fraction of collagen in solution (FIG. 5A), which indicates that the DNA is distributed amongst the collagen.

### Example 5: Aptamer Predicted Structures

Materials and Methods

Experimental Conditions

Calculated using mFold hosted by The RNA Institute at the University of Albany SUNY. Conditions for calculations were 25° C., 10 mM [Na+], oligomer corrected.

Results

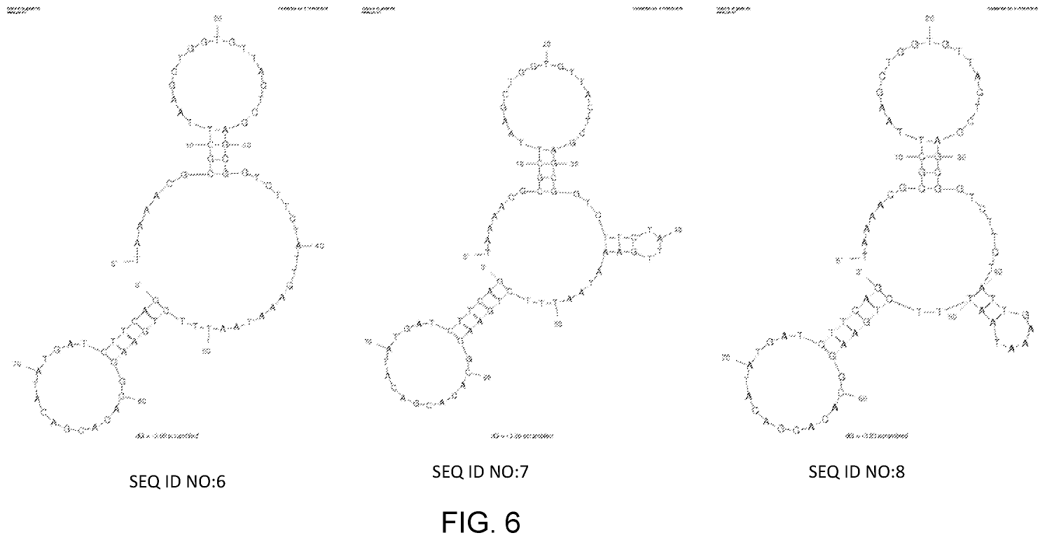

Three thermodynamically stable configurations were found (SEQ ID NOs:6-8). Two had hairpins, and 1 had 2 hairpins (FIG. 6).

### Example 6

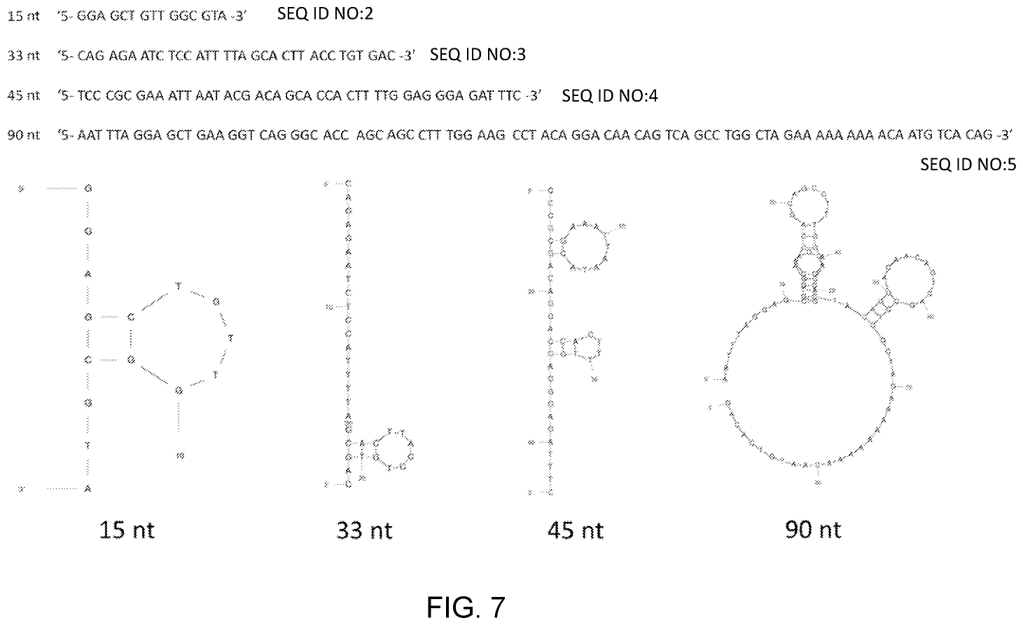

FIG. 7 shows sequences and predicted structures of random 15, 33, 45, and 90 nucleotide (nt) ssDNA oligomers (SEQ ID NOs:2-5, respectively). Lowest energy predicted structures were calculated using the mFold web server.

FIGS. 8A to 8C ssDNA oligomers with 15, 33, 45, and 90 nucleotides (nt) and their binding to type I collagen. ssDNA binding to collagen measured as the mass of bound DNA per mass of collagen as a function of mass fraction of DNA in solution (FIG. 8A). ssDNA binding to collagen measured as the moles of bound DNA per mass of collagen as a function of mass fraction of DNA in solution (FIG. 8B). The horizontal bars in (FIG. 8B) represent the range of DNA mass fraction where fiber formation was observed, from the top oligomers were 15, 33, 45 and 90 nt, respectively. When value for maximum binding from (FIG. 8B) of each oligomer was plotted against the inverse of the oligomer molecular weight, the data followed a linear relationship with R²>0.95 (FIG. 8C). ssDNA binding was measured in triplicate. Data is presented as mean±standard deviation.

At first, ssDNA oligomer length appeared to have no effect when measured as the amount of bound DNA per available collagen on a mass per mass basis (FIG. 8A). ssDNA oligomer binding peaked at ˜0.15 μg ssDNA/μg collagen which occurred between 12-18% mass fraction of DNA in solution. Interestingly, the 90 nucleotide ssDNA oligomer displayed reduced binding with increasing mass fraction of DNA in solution after its maximum binding. However, when measured as the amount of bound DNA per available collagen on a mole per mass basis the effect of length was revealed (FIG. 8B). The shorter the ssDNA oligomer, the more molecules of ssDNA would complex with a given mass of collagen. We evaluated the trend using the maximum amount of bound ssDNA per amount of collagen. (FIG. 8B). The maximum binding followed an inverse relationship with ssDNA oligomer molecular weight (length), reinforcing that shorter ssDNA has an avidity for binding with collagen (FIG. 8C).

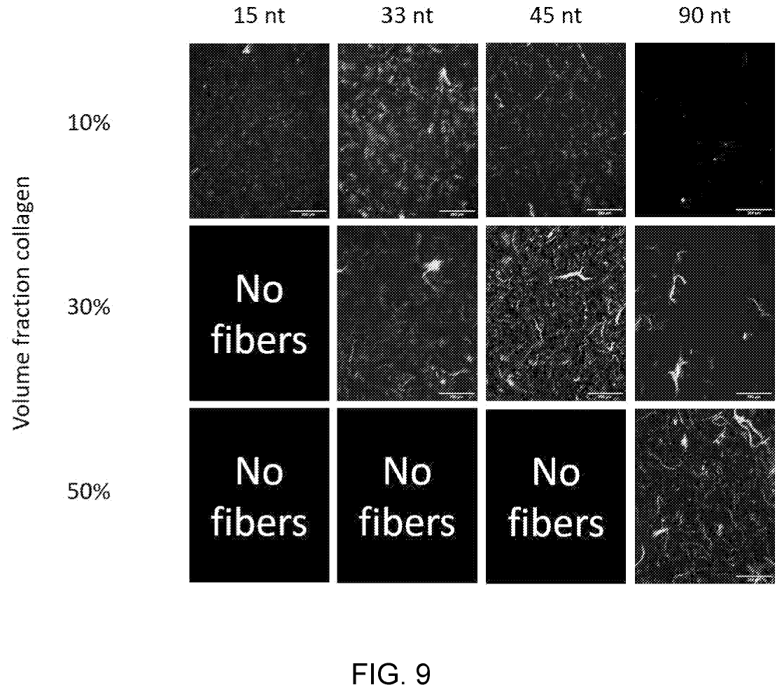

FIG. 9 shows representative fluorescence microscopy images of immobilized ssDNA-collagen fibers formed ssDNA with lengths of 15, 33, 45, and 90 nucleotides (nt) and different volume fractions of collagen. ssDNA in the fibers was fluorescently labeled using SYBR Safe DNA stain.

Fibers formed with varying density and size distribution for different volume fractions of collagen, favoring volume fractions of collagen that equated to mass fractions of DNA in solution in the range of 8-30%. This corresponded to DNA-collagen binding greater than 0.05 μg bound ssDNA/μg collagen. However, as shown by the 90 nucleotide ssDNA, there is an optimal range for fiber formation. For a mass fraction of DNA in solution of ˜45%, no fibers were observed; instead, a few faint ssDNA rich globs were present potentially the result of ssDNA self-aggregation and/or a lack of sufficient collagen in solution.

### Example 7

Bio-applications of multiferroics provide a minimally invasive approach to induce electric fields in vivo. E-fields in biology are typically range of 0-10 V/cm and cell membranes up to 10⁵ V/cm. H-fields between 5-10 kA/m (˜6-12 mT) will produce biologically relevant fields. There is a need to find a way to fabricate multiferroics that can be used in vivo.

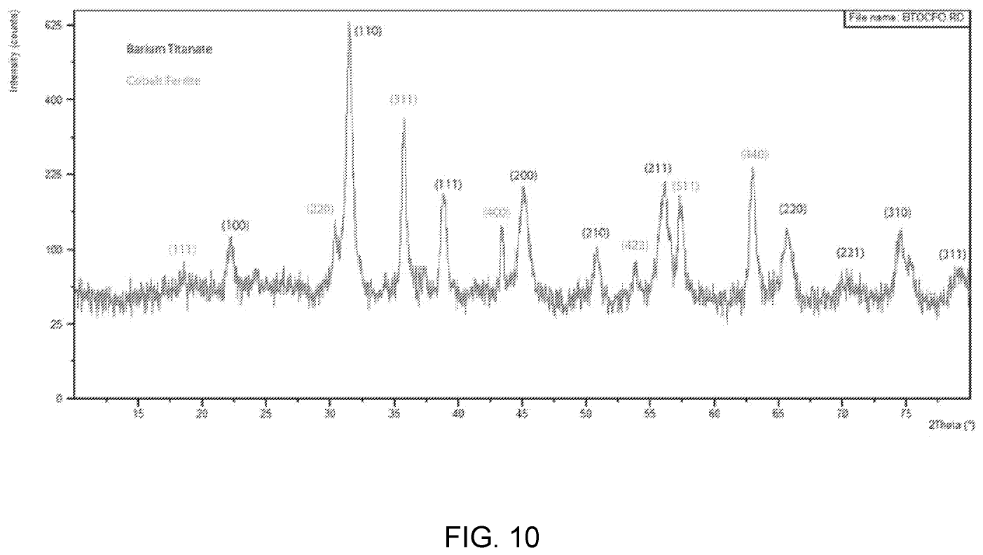

Two such fabrications can comprise electrospinning biphasic fibers and forming: polymer composites and ceramic composites. Electrospun ceramic composites can comprise barium titanate and cobalt ferrite (can be 1:1), and magnetoelectric janus fibers (also referred to herein as magnetoelectric composite materials) can be created. Properties of such materials are shown in FIG. 10.

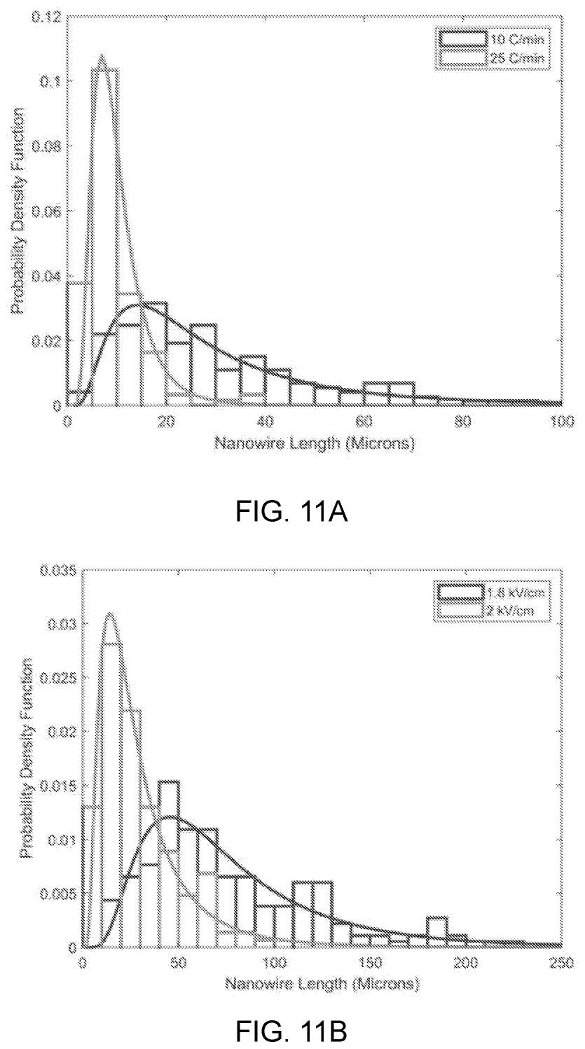

During fabrication, both calcination ramp rate and/or electrospinning voltage can be used to control nanowire length (FIGS. 11A-11B). The final nanowire length can be proportional to the as-spun fiber diameter.

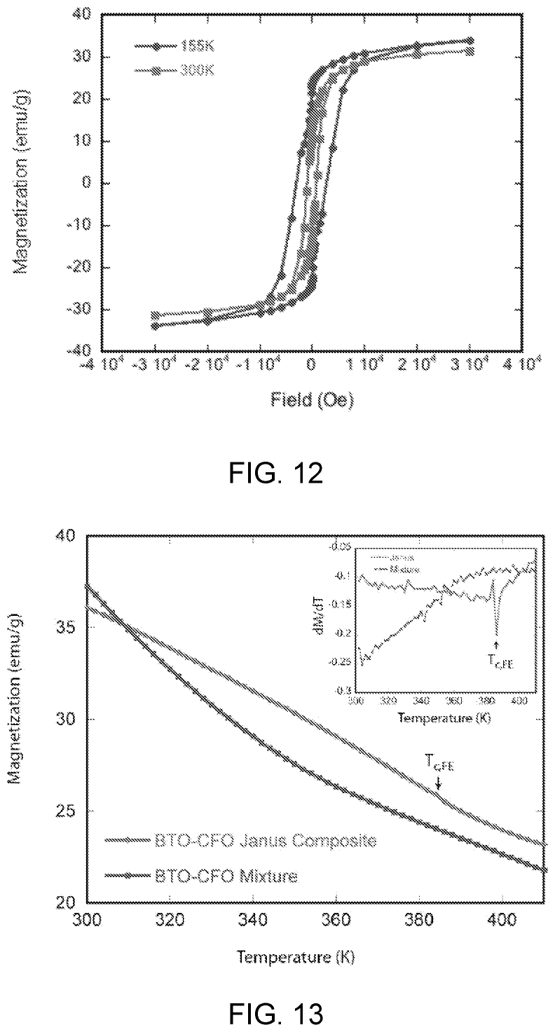

Ferrimagnetic properties of the BaTiO3-CoFe2O4 composites are confirmed through magnetization measurements (FIG. 12). The saturation magnetization of the composite is ˜43 wt. % of pure cobalt ferrite fibers.

Anomalous magnetization behavior at the ferroelectric Curie temperature is indicative of magnetoelectric coupling between the two phases (FIG. 13).

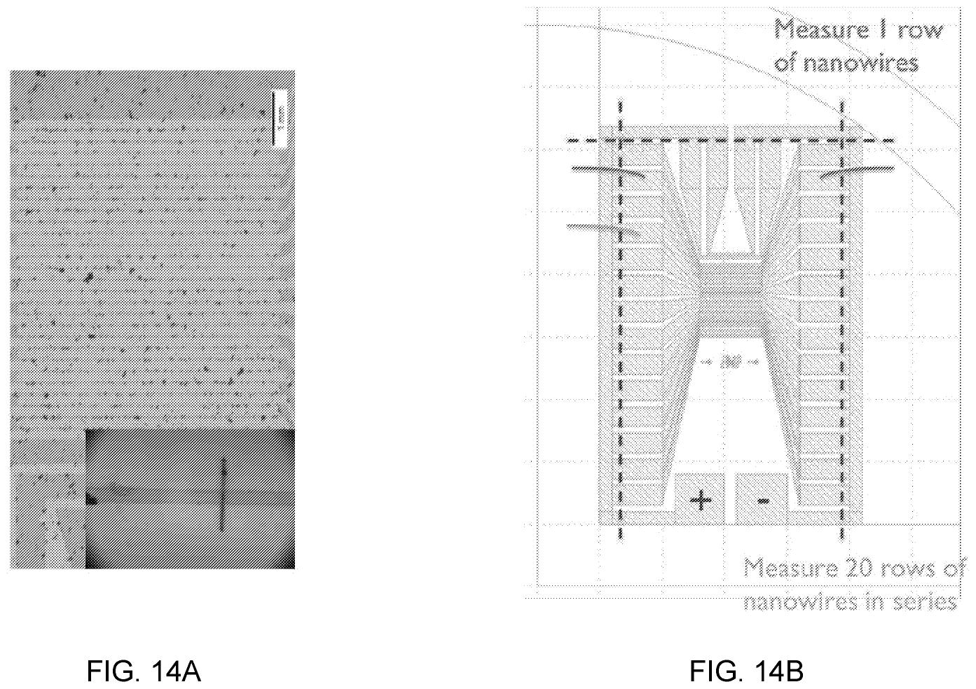

Such fibers can be fabricated in arrays and the magnetoelectro coefficient thereof measured (FIGS. 14A-14B).

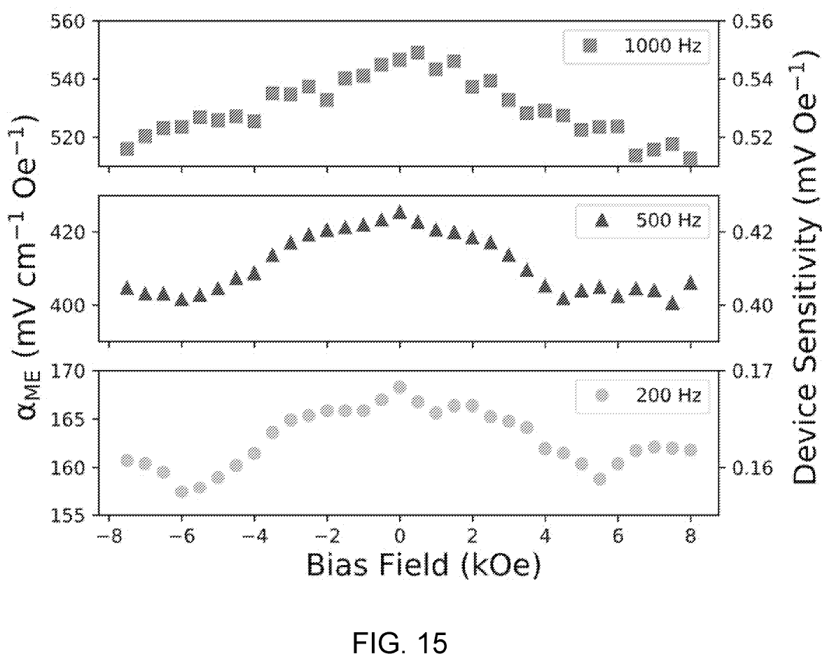

Lock in magnetoelectric measurements: zero bias magnetoelectric effect can be attributed to self-biasing of the nanowires by the magnetic phase (FIG. 15). Increasing magnetoelectric effect with frequency is attributed to a decrease in leakiness of the ferrite.

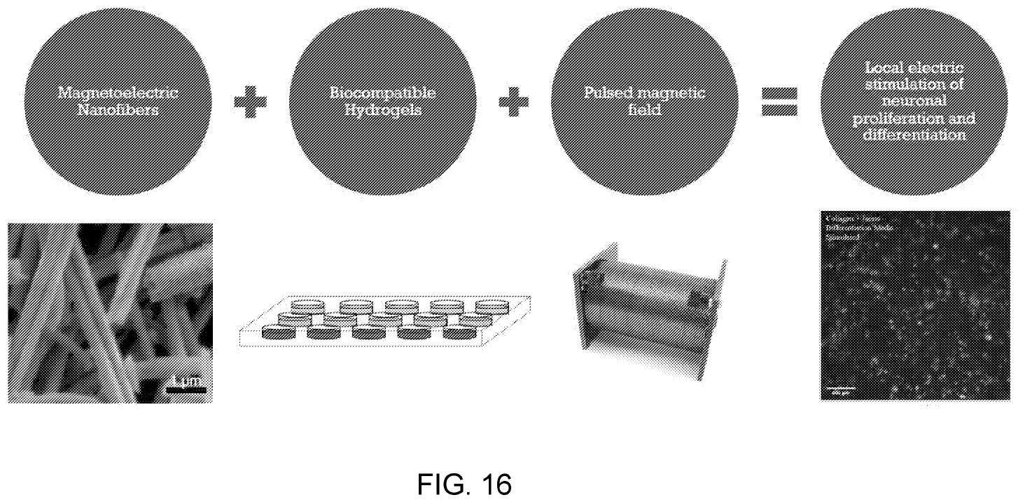

Such materials can be used for cell culture in vitro, to increase process length of cells such as a neurons, for example (embodiment of such shown in FIG. 16).

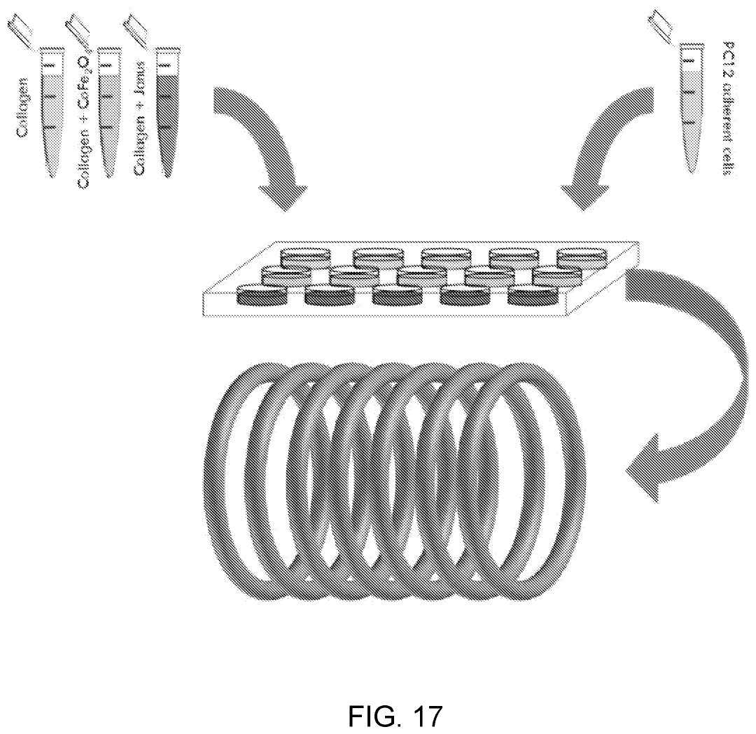

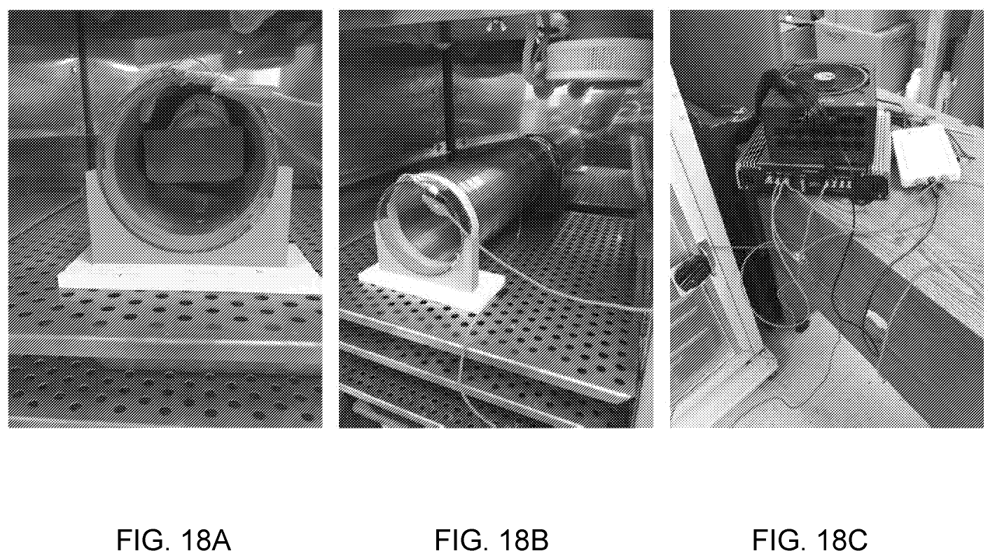

Magnetoelectric stimulation can be applied to the materials and cells in vitro (FIG. 17). Magnetoelectric fibers can be dispersed in collagen hydrogels and used for tissue culture. PC12 neuronal-like cells were used to study effects of ME stimulation on neuronal growth. Effects of ME stimulation were studied using n LDH (lactate dehydrogenase) for cytotoxicity; PicoGreen for proliferation; and fluorescent confocal microscopy for differentiation. Set-up for magnetoelectric stimulation is shown in FIGS. 18A-18C.

All fields are applied at a frequency of 60 Hz. Magnetoelectric (ME) coefficient used for calculations; 180 mV/cm Oe; two of the trials suggest an upper limit above which cells are not proliferating.

TABLE 1
Magnetoelectric Stimulation Regimes

| | | Calculated | |
|---|---|---|---|
| Voltage | | Electric field | |
| Applied to | Magnetic Field | from ME Effect | |
| Solenoid (V) | Strength (mT) | (mV/mm) | Time in Field (hrs) |
| 20 | 1.1 | 199-201 | 1 |
| 20 | 1.1 | 199-201 | 4 |
| 30 | 1.6 | 298-302 | 1 |
| 30 | 1.6 | 298-302 | 3 |
| 40 | 2.2 | 398-402 | 1 |

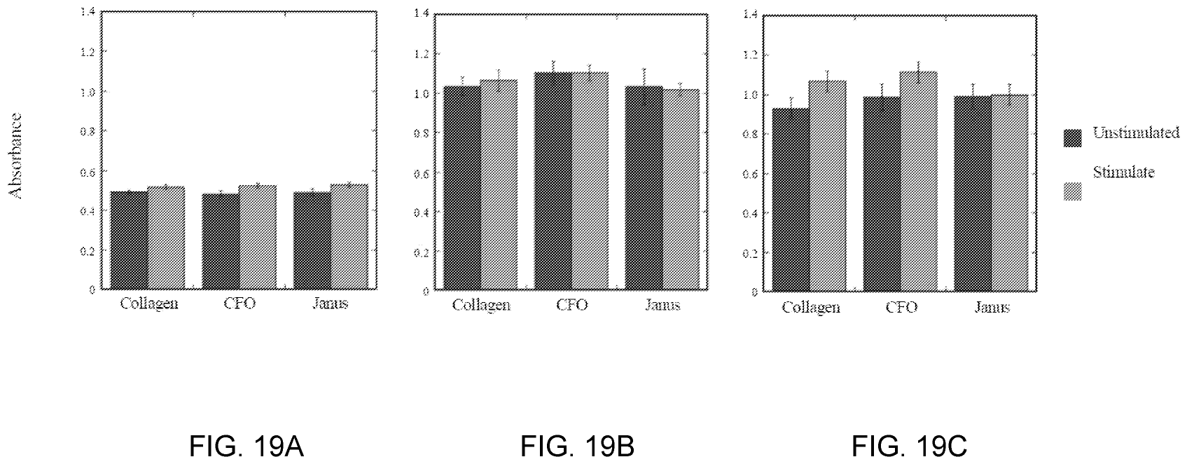

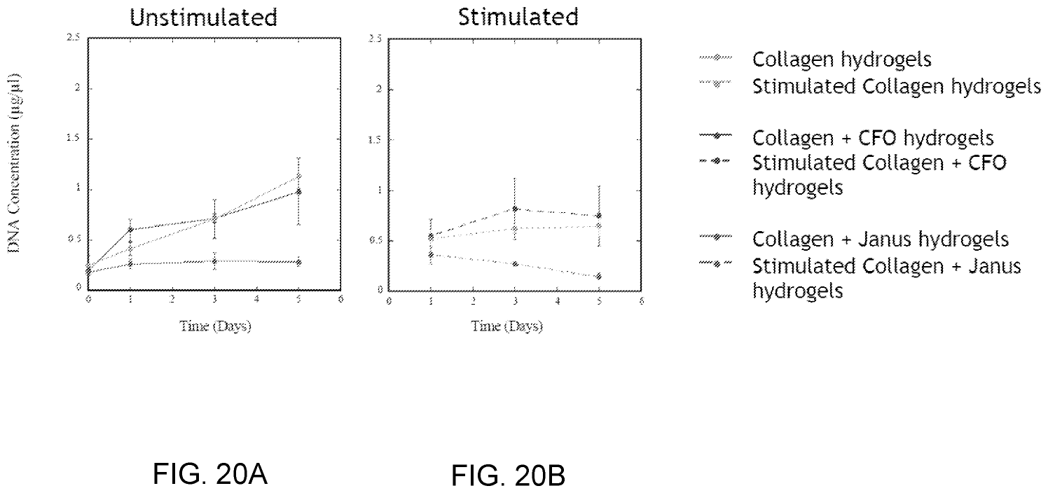

Toxicity studies were performed to examine the effect of magnetoelectric stimulation on toxicity (FIGS. 19A-19C and 20A-20B). Cytotoxicity studied with LDH assays; Collagen is cytocompatible; and stimulation performed at ˜200 mV/mm, 1 hour/day. Minimal cytotoxic effects from CoFe₂O₄(CFO) or Janus hydrogels, or magnetic stimulation. Furthermore, no significant effects on proliferation from magnetoelectric stimulation were observed in PicoGreen proliferation studies.

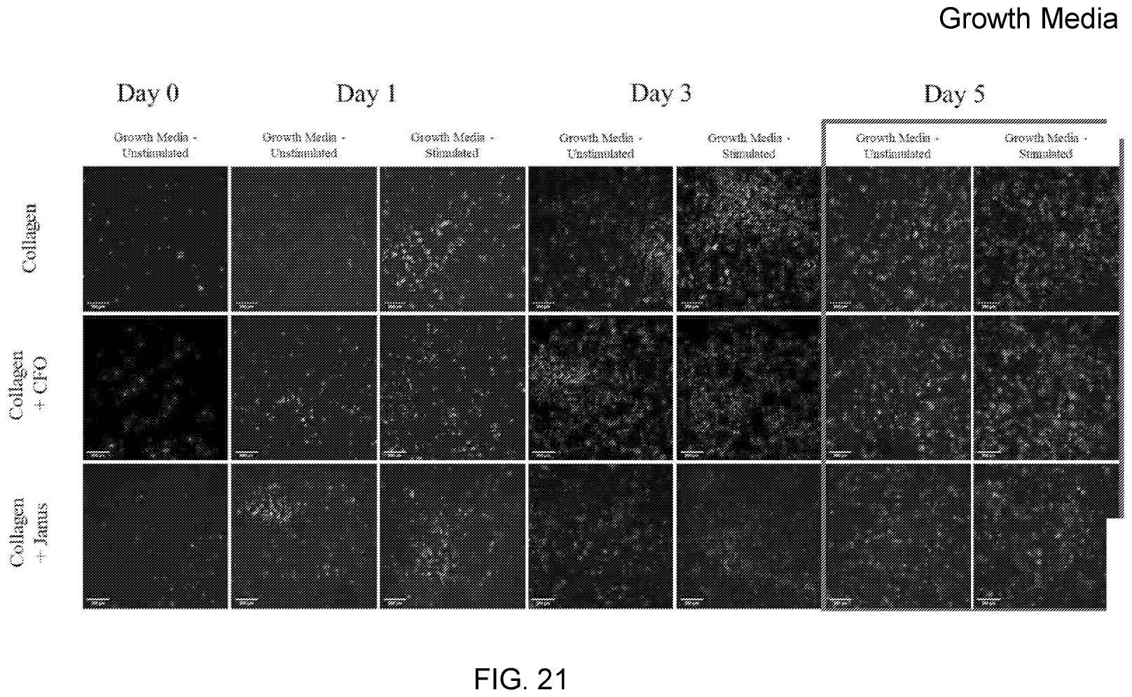

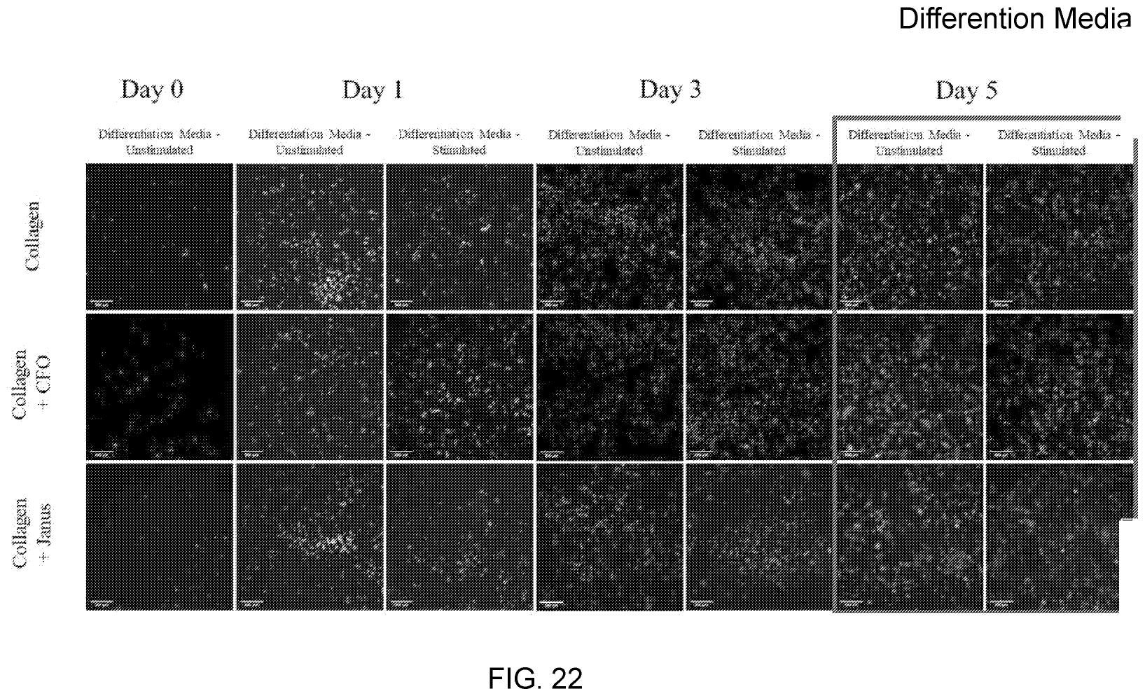

The effect of magnetoelectric stimulation on proliferation (FIG. 21) and differentiation (FIG. 22) was observed. Cells were stimulated for 1 hour per day in a 1.1 mT magnetic field; ˜200 mV/mm; varying growth conditions: Growth media—unstimulated, Growth media—stimulated, differentiation media (addition of NGF) —unstimulated, differentiation media (addition of NGF) —stimulated; NGF=nerve growth factor, which chemically induces differentiation. Fluorescent confocal microscopy allowed imaging of the cells through the hydrogels. Cells were stained with fluorescent CellTracker® Green. The following results were observed:

TABLE 2
Neurite outgrowth at day 5

| | % of Cells |
|---|---|
| | Exhibiting Neurites |
| Collagen + Janus − Differentiation Media - | 5.6% |
| Unstimulated | |
| Collagen + Janus − Differentiation Media - | 4.1% |
| Stimulated | |

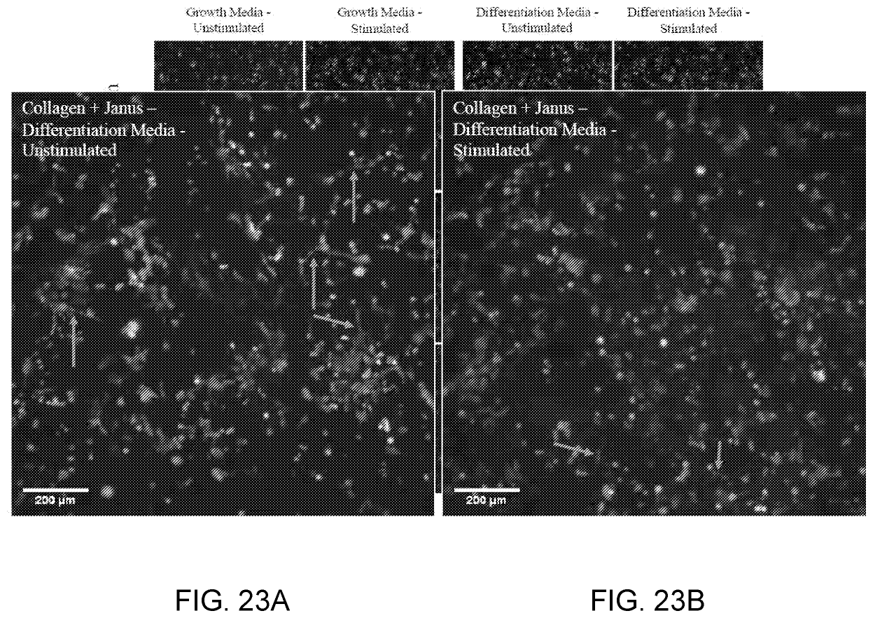

The effect of magnetoelectric stimulation on neurite outgrowth was further observed. The beginnings of neurite extension is seen in samples at 5 days of growth/stimulation (FIGS. 23A-23B, FIG. 25). Additional studies are necessary to fully elucidate the potential of magnetoelectric stimulation of neurites.

TABLE 3
Neurite outgrowth at 5 days of growth/stimulation.

| | Growth | Growth | Differentiation | Differentiation |
|---|---|---|---|---|
| | Media- | Media- | Media- | Media- |
| | Unstimulated | Stimulated | Unstimulated | Stimulated |
| Collagen | 4.0 +/− 1.3% | 1.3 +/− 1.0% | 4.4 +/− 1.8% | 1.8 +/− 1.1% |
| Collagen + | 7.8 +/− 6.2% | 2.9 +/− 1.9% | 8.8 +/− 6.9% | 6.5 +/− 5.5% |
| CoFe₂O₄ | | | | |
| Collagen + Janus | 5.1 +/− 2.1% | 3.1 +/− 0.9% | 5.6 +/− 5.1% | 4.3 +/− 0.9% |

Gels and magnetic/magnetoelectric stimulation induce minimal cytotoxicity. No significant difference in proliferation between stimulated and unstimulated samples. Imaging of cells through the gels was able to be achieved using fluorescent confocal microscopy. The beginnings of neurite extension is seen in samples at 5 days of growth/stimulation. Longer studies will be necessary to elucidate the effects of magnetoelectric stimulation.

Summary:

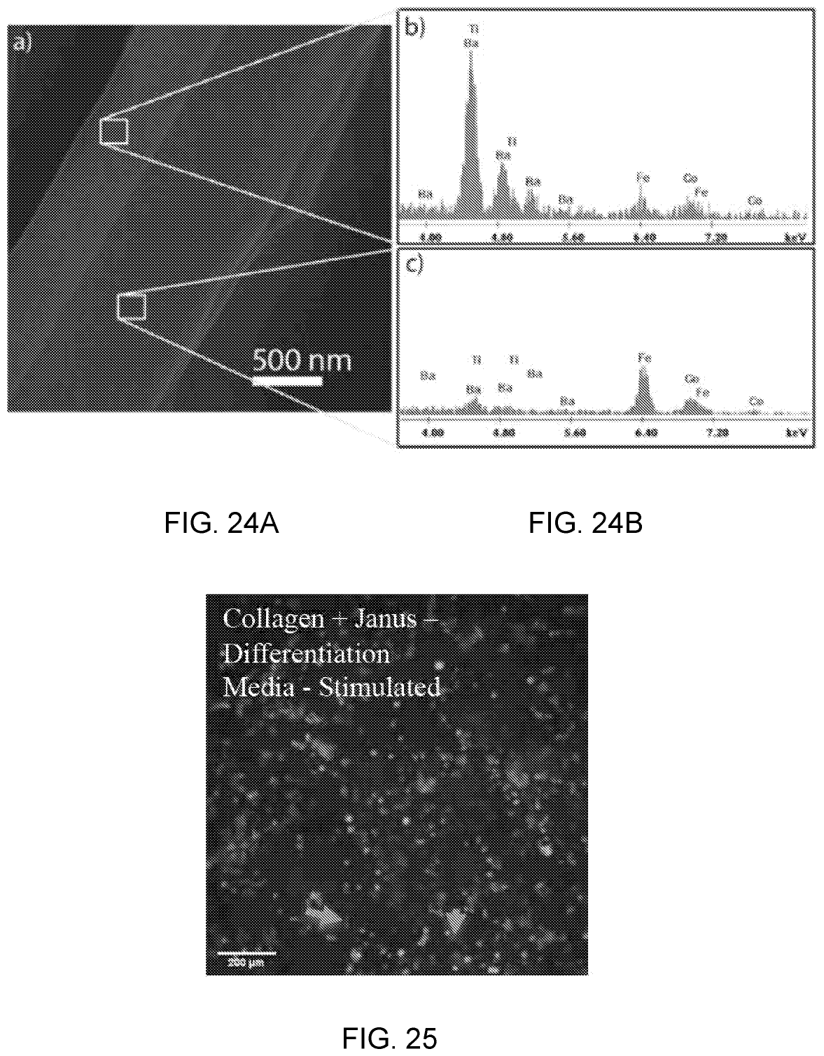

Demonstrated the ability to co-electrospin biphasic Janus type magnetoelectric materials (aspects of such shown in FIGS. 24A-24B). Developed methods to perform magnetoelectric measurements on arrays of nanofibers versatile platform to make bi- and tri-phasic oxide materials. Composites on a particle or fiber provide a route to a new class of magnetoelectric biomaterial.

### Example 8

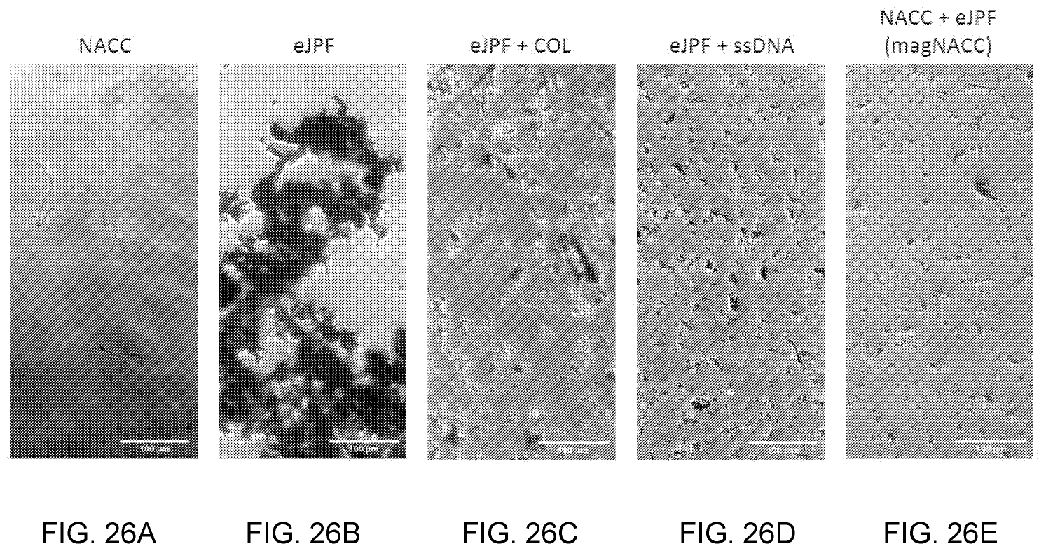

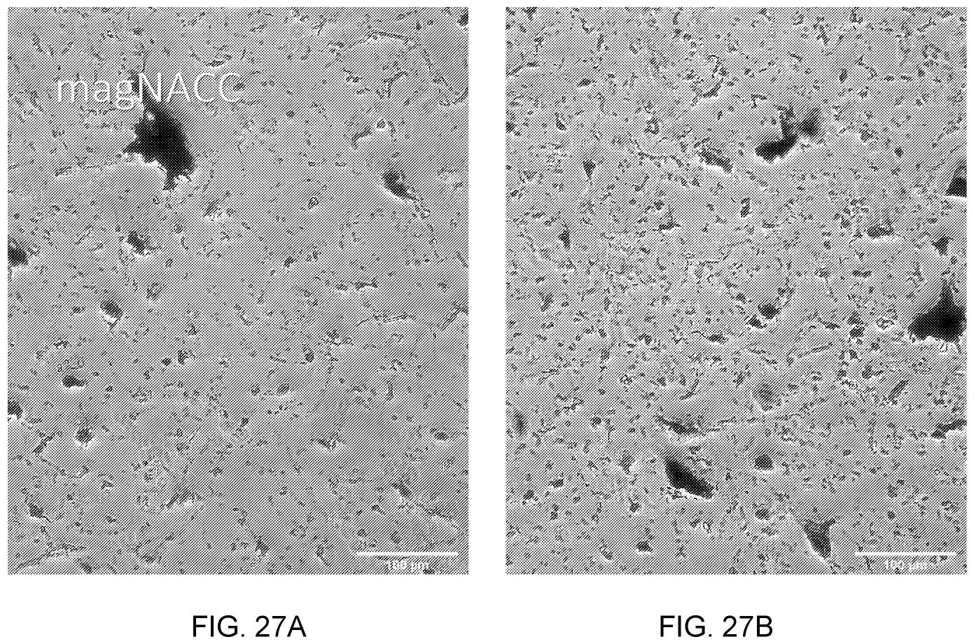

Methods:
Ground up electrospun Janus fiber mat (eJPF) with mortar and pestle to prepare particulate fibers to make BTO-CFO janus particulate
   Resuspended 22.1 mg in 1000 μL deionized water
   Serial diluted at half dilutions to make 690 μg/mL solution
Combined 60 μL of 300 μg/mL type I rat tail collagen (COL) with 126 μL of 690 μg/mL BTO-CFO janus particulate and then added 14 μL of 10 μM 80 nt random sequence ssDNA (ssDNA) to yield a final concentration of 90 μg/mL COL, 0.7 μM ssDNA, and 435 μg/mL BTO-CFO janus particulate
Controls included substituting each component with the same volume of deionized water as the replaced constituent
Prepared in microfuge tubes and vortex for several seconds to mix 14 μL aliquots placed on glass slide and imaged by inverted phase contrast microscope (FIGS. 26A-26E; 27A-27B).

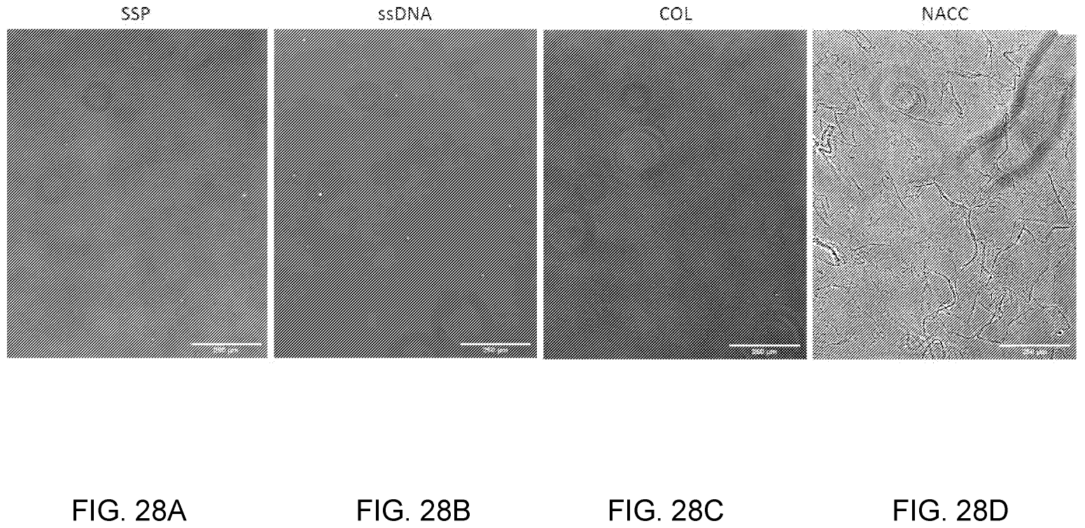

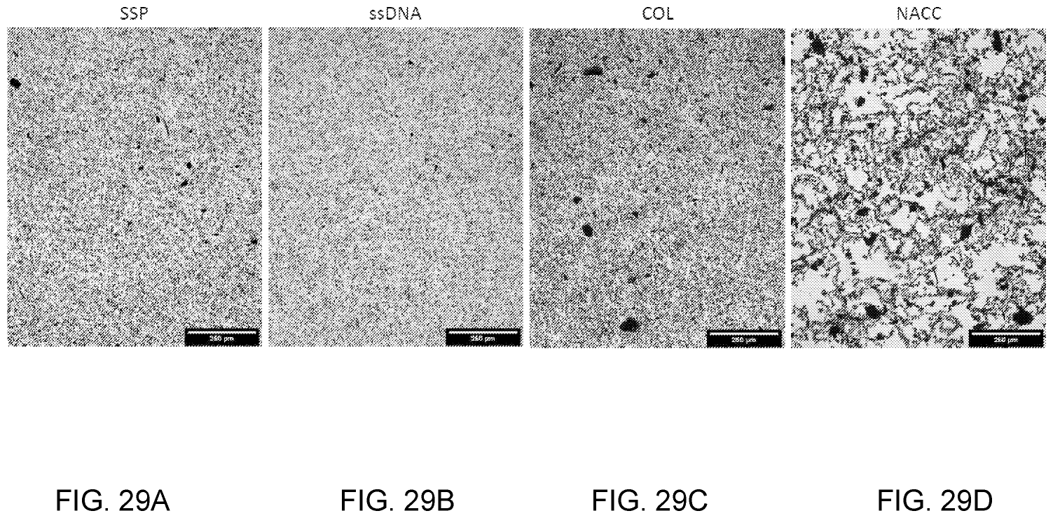

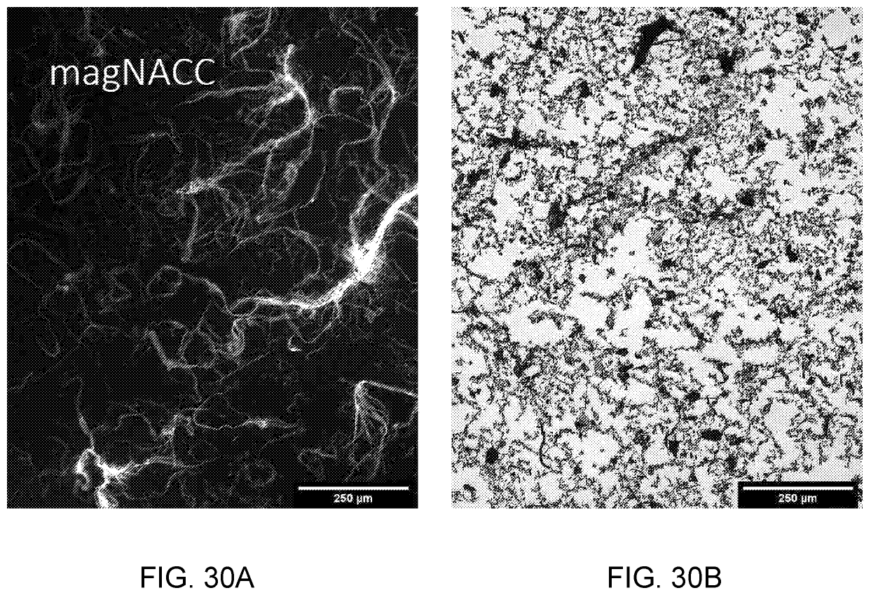

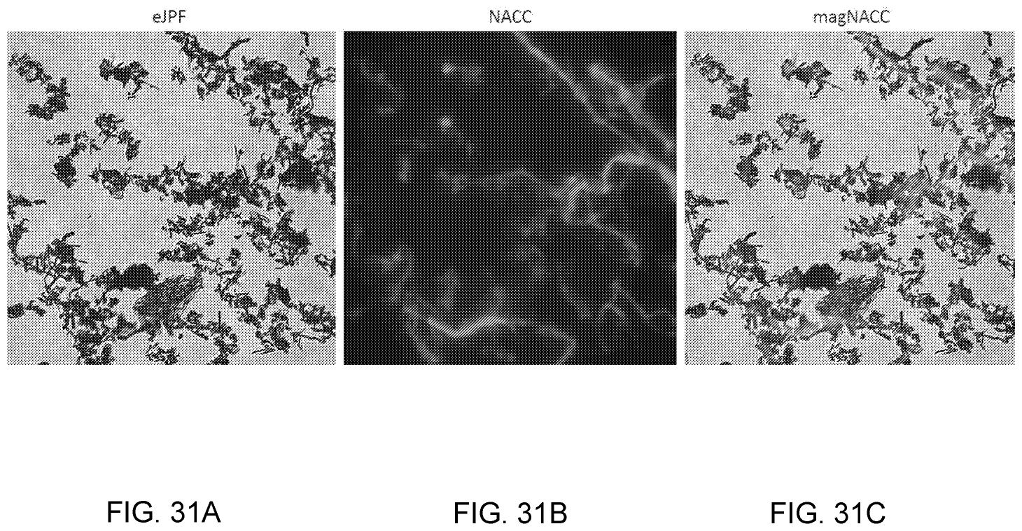

Methods
Ground up electrospun Janus fiber mat (eJPF) with mortar and pestle to prepare particulate fibers to make BTO-CFO janus particulate
   Resuspended 22.1 mg in 1000 μL deionized water
   Serial diluted at half dilutions to make 690 μg/mL solution
Combined 300 μg/mL type I rat tail collagen (COL) and 1 μM 40 nt (ssDNA) to yield a final concentration of 60 μg/mL COL, 0.8 μM ssDNA
Immobilized on sulfo-SANPAH (SSP) treated tissue culture polystyrene surface
Incubated in 434 μg/mL eJPFsolution for 10 minutes
Rinsed with deionized water 3 times
Incubated with SYBR Safe DNA stain for at least 30 minutes in dark
Imaged by both fluorescence and phase contrast microscopy to visualize surface without BTO-CFO janus particulate are shown with SSP, ssDNA, COL, and NACC (FIGS. 28A-28D) and surfaces with BTO-CFO janus particulate are shown with SSP, ssDNA, COL, and NACC (FIGS. 29A-29D)
Sulfo-SANPAH (SSP), ssDNA, and collagen (COL) treated surfaces showed no qualitative differences in coverage or BTO-CFO janus particulate distribution. All three retained a uniform bound surface layer of BTO-CFO janus particulate following rinsing. NACC surfaces showed non-uniform bound surface layer of BTO-CFO janus particulate following rinsing with bare regions devoid of BTO-CFO janus particulate, which was not observed in the other conditions (FIGS. 29A-29D)
The NACC fibers are confirmed to be covered and filled the BTO-CFO janus particulate as seen in FIGS. 30A-30B
FIGS. 31A-31C show that BTO-CFO janus particulate localize to NACC fibers. This triphasic composite we have termed magnetostrictive-piezoelectric NACCs or magNACCs

Possibilities for Interaction
The phosphate backbone of the DNA in NACCs is interacting with the divalent cationic species (Co²⁺, Ba²⁺) exposed at the surface of the BTO-CFO janus particulate
Similar interactions are known to exist with calcium phosphate phases e.g. hydroxyapatite and a number of other cationic species

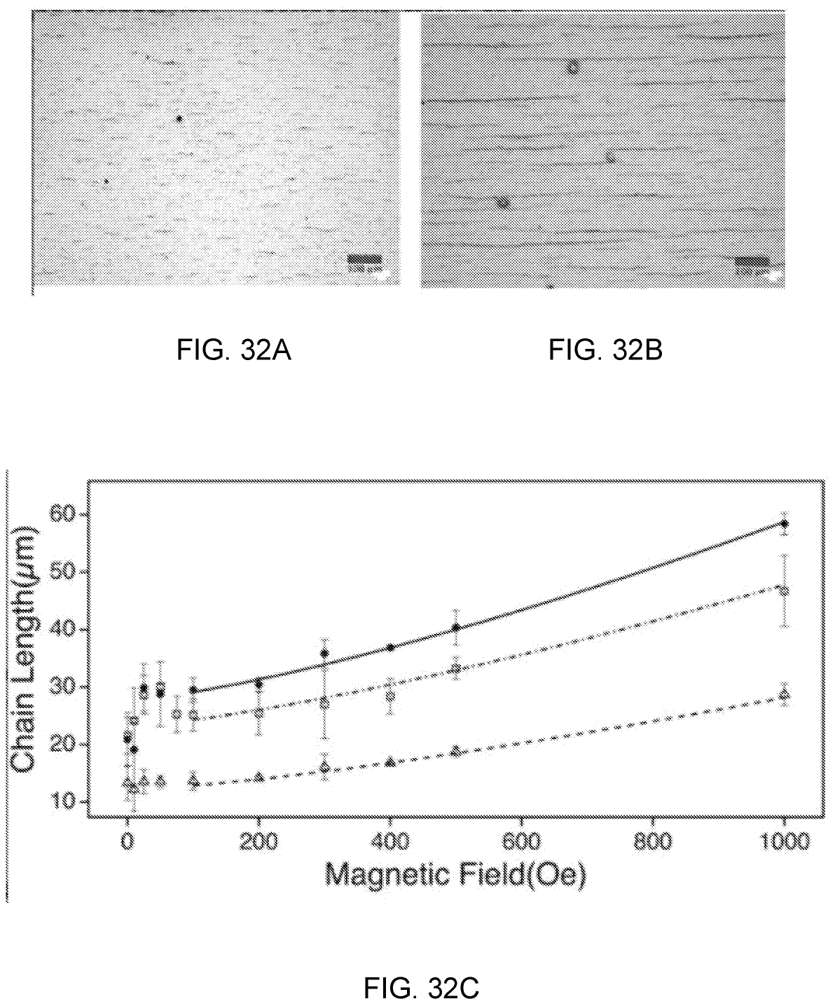

Additional Aspects (FIGS. 32A-32C)
Generate aligned magNACC fibers by applying a magnetic field during the immobilization step following work done by Andrew lab already for stimulating the aligned self-assembly of BTO-CFO janus particulate by an external magnetic field
Culture cells on aligned magNACCs to stimulate contact guided alignment/migration of cells as well as electric potential stimulation from BTO-CFO janus particulate If fruitful generate cross-patterning to mimic woven bone architecture
Cells according to this example (or other aspects of the present disclosure) could be fibroblasts, endothelial cells, bone cells, muscle cells, neural cells
Incorporate CaPO₄ platelets as well to have biomimetic bone composition

### Example 9

Additional aspects of DNA-collagen complexes as described herein can be found in PCT/US2020/048488 filed on Aug. 28, 2020, which is incorporated by reference in its entirety as if fully set forth herein.

### Example 10

Background: Nucleic acid-collagen complex (NACC) fibers and hydrogels self-assemble when single-stranded DNA (ssDNA) and type I collagen are mixed together¹. Hydrogels are used as constructs to engineer tissues. However, collagen hydrogels have poor mechanical properties. This is due to the random alignment of the fibers and the high-water content of the gels². While collagen fibers show changes in orientation due to mechanical deformation, elastin fibers tend to remain uniformly distributed³. In the extracellular matrix (ECM) elastin fibers impart a compressive intrinsic stress on collagen³. Therefore, if elastin is added to the NACC, then the mechanical properties of the complexes can be altered.

Methods: The NACCs were prepared by mixing 1 μM ssDNA with 0.3 mg/mL type I collagen. A separate solution was prepared adding 0.3 mg/mL elastin to the NACCs. The solutions were made with a 1:1 and 1:2 concentration ratio of elastin to collagen. A random 80 nucleotide ssDNA sequence was used because the sequence of the ssDNA does not affect the formation of the NACC fibers¹. To best visualize the fibers, they had to be immobilized onto a glass slide. This was done by treating a glass slide with (3-aminopropyl)triethoxysilane and then immobilizing the fibers using sulfo-SANPAH. The fibers were stained with SYBR Safe DNA stain to highlight the ssDNA in the fibers during fluorescence imaging. The Young's modulus of these fibers was measured by atomic force microscopy (BioAFM). A Bruker/JPK NanoWizard 4 BioAFM was used for these experiments. Fibers were measured in QI mode and force curves were fit to a Hertz model to extract the Young's modulus. When using a higher concentration of ssDNA (10 μM), collagen, (3.0 mg/mL) and elastin (3.0 mg/mL) NACC gels were formed. The storage modulus and the loss modulus of these gels were measured using an Anton Paar modular compact rheometer. The data collected was compared to that of NACC without elastin. Lastly, a DNA binding assay was conducted to assess the effect of elastin on the amount of ssDNA that binds with the collagen¹

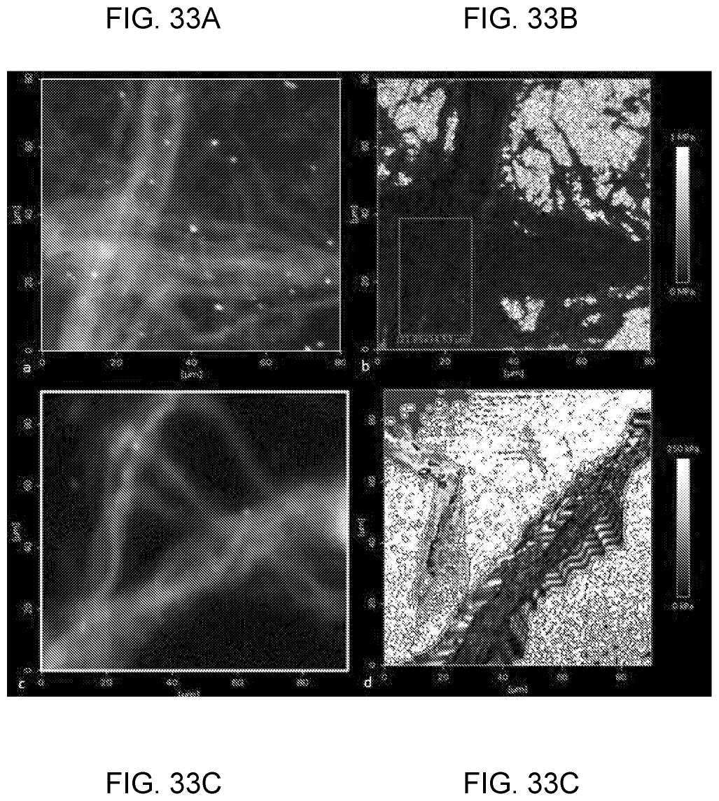

Results: As measured by BioAFM, the Young's modulus was lower in the fibers with the elastin than those without (FIG. 33A, FIG. 33C). The Young's modulus of the fibers with elastin averaged to 35 kPa±24 kPa (FIG. 33B). The Young's modulus for the fibers without elastin averaged to 59 kPa±21 kPa (FIG. 33D). Meaning there was a significant change in mechanical properties by adding the elastin. The p-value was less than 0.0001, meaning it is extremely statistically significant. By using the BioAFM, fluorescence images of the fibers were taken at the same location of the AFM images allowing for the correlation between the features of the fibers and the mechanical properties of the fibers to be made. The NACC fibers and NAECC fibers had a great variance in terms of the mechanical properties. As for the NACC and NAECC gels a rheometer was used to measure the storage modulus and the loss modulus of the gel. Ratio between the loss modulus and storage modulus was calculated. The NAECC gel with a 1:1 concentration of collagen to elastin, has similar elastic behavior as the NACC gel. The DNA binding assay showed that there was more unbounded DNA in the solutions without elastin than those with elastin. Meaning more DNA was bound to collagen in the presence of elastin. In the solution of just collagen and DNA there was 0.132 μM of unbound DNA, while in the solution with a 1:1 concentration of elastin to collagen there was 0.077 μM of unbound DNA and in the solution with a 1:2 concentration elastin to collagen there was 0.116 μM of unbound DNA. The changes in concentration of unbound DNA indicate that the collagen and elastin interacted.

Conclusions: Collagen and elastin naturally interact in the ECM. By being able to adjust the mechanical properties of the hydrogels with the inclusion of elastin, different components can be mimicked such as a singular alveolar wall⁴. This takes advantage of the natural interactions between elastin, DNA, and collagen, and allows for a simpler method to integrate elastin to NACCS. Furthermore, by adding DNA to these complexes, specific biomolecules or cells could be targeted using a DNA aptamer. The nucleic acid elastin collagen complexes with allow for further development and applications of hydrogels by being able to adjust the properties of the gels.

### REFERENCES FOR EXAMPLE 10

1. James, B. D., Saenz, S., van Gent, A., Allen, J. B., ACS Biomater. Sci. Eng. 2020, 6, 213-218 (2019).
2. M. Ahearne, Y. Y et al, Topics in Tissue Engineering, Vol. 4. Eds. N Ashammakhi, R Reis, & F Chiellini (2008).
3. Chow, M. J. et al, Biophysical Journal Vo. 106, 12, 2684-2692 (2014).
4. Dunphy, S. E., Journal of the Mechanical Behavior of Biomedical Materials, Volume 38, 251-259 (2014).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising
a collagen material; and
a magnetoelectric composite material;
wherein the collagen material comprises collagen fibers crosslinked with a plurality of DNA aptamers.

2. (canceled)

3. The composition of claim 1, wherein at least one of the one or more DNA aptamers selectively binds a growth factor or cytokine.

4. The composition of claim 1, wherein at least one of the plurality of DNA aptamers selectively binds a cell receptor.

5. The composition of claim 4, wherein the cell surface receptor is a signaling receptor, a stem cell receptor or growth factor receptor.

6. The composition of claim 1, wherein at least one of the plurality of DNA aptamers selectively binds an extracellular matrix protein.

7. The composition of claim 1, wherein the DNA aptamers comprise from 15 to 100 nucleotides.

8. The composition of claim 1, wherein the DNA aptamers comprise from 1 to 5 stem loops.

9. The composition of, wherein the plurality of DNA aptamers comprises 2 to 4 DNA aptamer sequences connected by a linker molecule to form an aptamer assembly.

10. The composition of claim 1, wherein the collagen comprises type I collagen, type II collagen, type Ill collagen, type V collagen, type XI collagen, or any combination thereof.

11. The composition of claim 1, wherein the magnetoelectric composite material comprises a piezoelectric composite material or a magnetorestrictive compositive material, individually or in combination.

12. The composition of claim 1, wherein the magnetoelectric composite material comprises BaTiO₃ and CoFe2O₄.

13. The composition of claim 1, wherein the magnetoelectric composite material comprises electrospun fibers.

14. The composition of claim 13, wherein the electrospun fibers have a length of about 1 μm to about 100 μm.

15. The composition of claim 13, wherein the electrospun fibers have a diameter of about 100 nm to about 1000 nm.

16. A method of tissue culture, comprising:
providing a composition of claim 1;
culturing one or more cells in the presence of the composition, a magnetic field, and an electric field.

17. The method of claim 16, wherein the magnetic field has a field strength of about 0 mT to about 5 mT.

18. The method of claim 16, wherein the electric field has a field strength of about 0 to about 500 mV/mm.

19. The method of claim 16, wherein the one or more cells comprise neuronal cells, neuronal progenitor cells, epithelial cells, fibroblasts, osteoclasts, osteoblasts, or muscle cells.

20.-34. (canceled)

35. The composition of claim 1, further comprising elastin.

36. The composition of claim 35, wherein elastin is present in a ratio to collagen of about 1:1 to about 1:2.

37.-40. (canceled)


SEQUENCE LISTING (8 sequences)

SEQ ID NO: 1 - DNA, 80 bp, Artificial sequence
  Synthetic construct
taaaacgcgcttaagctggtgttactcgagcggtcttctattgaaataatttctgaaggcacacgacatatgatcttcag

SEQ ID NO: 2 - DNA, 15 bp, Artificial sequence
  Synthetic construct
ggagctgttggcgta

SEQ ID NO: 3 - DNA, 33 bp, Artificial sequence
  Synthetic construct
cagagaatctccattttagcacttacctgtgac

SEQ ID NO: 4 - DNA, 45 bp, Artificial sequence
  Synthetic construct
tcccgcgaaattaatacgacagcaccacttttggagggagatttc

SEQ ID NO: 5 - DNA, 90 bp, Artificial sequence
  Synthetic construct
aatttaggagctgaaggtcagggcaccagcagcctttggaagcctavaggacaacagtcagcctggctagaaaaaaaaacaatgtcacag

SEQ ID NO: 6 - DNA, 80 bp, Artificial sequence; identical to SEQ ID NO: 1
  Synthetic construct

SEQ ID NO: 7 - DNA, 80 bp, Artificial sequence
  Synthetic construct
taaaacgcgcttaagctggtgttactcgagcggtcttctattgaaataatttctgaagcgacacgacatatgatcttcag

SEQ ID NO: 8 - DNA, 80 bp, Artificial sequence; identical to SEQ ID NO: 1
  Synthetic construct